(19) United States
(12) Patent Application Publication
(10) Pub. No.: US 2023/0059690 A1
(43) Pub. Date: Feb. 23, 2023

(54) ANTI-PSMA ANTIBODIES, ANTIBODY DRUG CONJUGATES, AND METHODS OF USE THEREOF

(71) Applicant: CYTOMX THERAPEUTICS, INC., South San Francisco, CA (US)

(72) Inventors: Rene Hubert, South San Francisco, CA (US); Christopher Kemball, South San Francisco, CA (US); Pia Challita-Eid, South San Francisco, CA (US)

(21) Appl. No.: 17/790,842

(22) PCT Filed: Jan. 6, 2021
(86) PCT No.: PCT/US2021/012379

§ 371 Date: Jul. 5, 2022

Related U.S. Application Data
(60) Provisional application No. 62/957,780, filed on Jan. 6, 2020.

(51) Int. Cl. C07K 16/30; A61K 47/68; A61K 39/395; A61P 35/00; A61P 15/00

(52) U.S. Cl. CPC C07K 16/3069; A61K 47/6869; A61K 47/6811; A61K 39/39558; A61P 35/00; A61P 15/00; A61K 2039/505

(57) ABSTRACT

The invention relates generally to antibodies that bind PSMA, and methods of making and using these anti-PSMA antibodies in a variety of therapeutic, diagnostic and prophylactic indications.

### RELATED APPLICATIONS

This application claims the benefit of U.S. Provisional Application No. 62/957,780, filed Jan. 6, 2020, the contents of which are incorporated herein by reference in its entirety.

### REFERENCE TO SEQUENCE LISTING

The Sequence Listing submitted electronically concurrently herewith pursuant 37 C.F.R. § 1.821 in computer readable form (ASCII format) via EFS-Web as file name CYTX 077 PCT ST25.txt is incorporated herein by reference. The ASCII copy of the Sequence Listing was created on Jan. 6, 2021 and is 48 kilobytes in size.

### FIELD OF THE INVENTION

The invention relates generally to antibodies and antibody drug conjugates that bind PSMA, and the use of these anti-PSMA antibodies and antibody drug conjugates in a variety of therapeutic, diagnostic and prophylactic indications.

### BACKGROUND OF THE INVENTION

Antibody-based therapies have proven effective treatments for several diseases. In some cases, antibodies have found additional usefulness by conjugating them to agents, such as cytotoxic compounds. Such conjugated antibodies, also known as antibody drug conjugates (ADCs) allow the target-specific delivery of the conjugated toxin to cells or tissues that express the target of the antibody. In this manner, the ADC provides a way to specifically deliver a cytotoxic compound based on the antibody specificity.

Accordingly, there is a continued need in the field of new antibodies and ADCs to new molecular targets.

SUMMARY OF THE INVENTION

The disclosure provides antibodies or antigen-binding fragments thereof that specifically bind PSMA, also known as Prostate-Specific Membrane Antigen, Folate Hyrdrolase 1 (FOLH1), Glutamate Carboxypeptidase 2 (GCP2), N-Acetylated-Alpha-Linked Acidic Dipeptidase I (NAALAD1), Pteroylpoly-Gamma-Glutamate Carboxypeptidase, Folylpoly-Gamma-Glutamate Carboxypeptidase, Cell Growth-Inhibiting Gene 27 Protein, Membrane Glutamate Carboxypeptidase, Glutamate Carboxypeptidase II, Glutamate Carboxylase II, EC 3.4.17,21, NAALAdase, FGCP, FOLK GCP2, MGCP, Folate Hydrolase (Prostate-Specific Membrane Antigen), N-Acetylated Alpha-Linked Acidic Dipeptidase 1, Prostate Specific Membrane Antigen Variant F, GCPII, and/or PSM. The use of the term “PSMA” is intended to cover any variation thereof, such as, by way of non-limiting example, FOLH1, and all variations are used herein interchangeably.

In some embodiments, the antibody includes an antibody or antigen-binding fragment thereof that specifically binds PSMA. In some embodiments, the antibody or antigen-binding fragment thereof that binds PSMA is a monoclonal antibody, domain antibody, single chain, Fab fragment, a F(ab′)₂ fragment, a scFv, a scAb, a dAb, a single domain heavy chain antibody, or a single domain light chain antibody. In some embodiments, such an antibody or antigen-binding fragment thereof that binds PSMA is a mouse, other rodent, chimeric, humanized or fully human monoclonal antibody.

In some embodiments, the antibody or antigen-binding fragment thereof comprises a heavy chain variable region amino acid sequence comprising SEQ ID NO: 31 or SEQ ID NO: 32. In some embodiments, the antibody or antigen-binding fragment thereof comprises a heavy chain variable region amino acid sequence comprising SEQ ID NO: 31. In some embodiments, the antibody or antigen-binding fragment thereof comprises a heavy chain variable region amino acid sequence comprising SEQ ID NO: 32.

In some embodiments, the antibody or antigen-binding fragment thereof comprises a light chain variable region amino acid sequence comprising SEQ ID NO: 29 or SEQ ID NO: 30. In some embodiments, the antibody or antigen-binding fragment thereof comprises a light chain variable region amino acid sequence comprising SEQ ID NO: 29. In some embodiments, the antibody or antigen-binding fragment thereof comprises a light chain variable region amino acid sequence comprising SEQ ID NO: 30.

In some embodiments, the antibody or antigen-binding fragment thereof comprises a heavy chain variable region amino acid sequence selected from the group consisting of SEQ ID NO: 31 and SEQ ID NO: 32, and a light chain variable region amino acid sequence selected from the group consisting of SEQ ID NO: 29 and SEQ ID NO: 30. In some embodiments, the antibody or antigen-binding fragment thereof comprises a heavy chain variable region amino acid sequence of SEQ ID NO: 31, and a light chain variable region amino acid sequence of SEQ ID NO: 29. In some embodiments, the antibody or antigen-binding fragment thereof comprises a heavy chain variable region amino acid sequence of SEQ ID NO: 32, and a light chain variable region amino acid sequence of SEQ ID NO: 30.

In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence comprising SEQ ID NO: 31 or SEQ ID NO: 32. In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence comprising SEQ ID NO: 32. In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence comprising SEQ ID NO: 31.

In some embodiments, the antibody or antigen-binding fragment thereof comprises a light chain variable region amino acid sequence that is at least 90%, 91%, 92%, 93%, 94%, 95%, 96%, 97%, 98% or 99% identical to an amino acid sequence comprising SEQ ID NO: 29 or SEQ ID NO: 30. In some embodiments, the antibody or antigen-binding fragment thereof comprises a light chain variable region amino acid sequence that is at least 90%, 91%, 9?%, 93%, 94%, 95%, 96%, 97%, 98% or 99% identical to an amino acid sequence comprising SEQ ID NO: 29. In some embodiments, the antibody or antigen-binding fragment thereof comprises a light chain variable region amino acid sequence that is at least 90%, 91%, 92%, 93%, 94%, 95%, 96%, 97%, 98% or 99% identical to the amino acid sequence SEQ ID NO: 30.

In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence selected from the group comprising SEQ ID NO: 31 or SEQ ID NO: 32, and a light chain variable region amino acid sequence that is at least 90%, 91%. 92%, 93%, 94%, 95%, 96%, 97%, 98% or 99% identical to an amino acid sequence selected from the group comprising SEQ ID NO: 29 or SEQ ID NO: 30.

In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence SEQ ID NO: 31, and a light chain variable region amino acid sequence that is at least 90%, 91%, 92%, 93%, 94%, 95%, 96%, 97%, 98% or 99% identical to an amino acid sequence comprising SEQ ID NO: 29.

In some embodiments, the antibody or antigen-binding fragment thereof comprises a heavy chain variable region amino acid sequence that is at least 90%, 91%, 92%, 93%, 94%, 95%, 96%, 97%, 98% or 99% identical to an amino acid sequence comprising SEQ ID NO: 32, and a light chain variable region amino acid sequence that is at least 90%, 91%, 92%, 93%, 94%, 95%, 96%, 97%, 98% or 99% identical to an amino acid sequence comprising SEQ ID NO: 30.

In some embodiments, the antibody or antigen-binding fragment thereof comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarily determining region 2 (VL CDR2, also referred to herein as CDRL2) sequence, and a variable light chain complementarity determining region 3 (VL CDR3, also referred to herein as CDRL3) sequence, wherein at least one complementarity determining region (CDR) sequence is selected from the group consisting of a VH CDR1 sequence comprising the amino acid sequence SYDMH (SEQ ID NO: 7) or NYWMS (SEQ ID NO: 10); a VH CDR2 sequence comprising the amino acid sequence VIWYDGSNKYYADSLKG (SEQ ID NO: 8) or NIKKDGSEKFYVDSVKG (SEQ ID NO: 11); a VH CDR3 sequence comprising the amino acid sequence VIAARTFYYYGMDV (SEQ ID NO: 9) or EIQLYLQH (SEQ ID NO: 12); a VL CDR1 sequence comprising the amino acid sequence RSSQSLLHSDGYNYLD (SEQ ID NO: 1) or RASQGISNWLA (SEQ ID NO: 4); a VL CDR2 sequence comprising the amino acid sequence LGSNRAS (SEQ ID NO: 2) or AASSLQS (SEQ ID NO: 5); and a VL CDR3 sequence comprising the amino acid sequence MQALQTPWT (SEQ ID NO: 3) or QQANSFPLT (SEQ ID NO: 6).

In some embodiments, the antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein at one complementarity determining region (CDR) sequence is selected from the group consisting of a VH CDR1 sequence comprising the amino acid sequence SYDMH (SEQ ID NO: 7); a VH CDR2 sequence comprising the amino acid sequence VIWYDGSNKYYADSLKG (SEQ ID NO: 8); a VH CDR3 sequence comprising the amino acid sequence VIAARTFYYYGMDV (SEQ ID NO: 9); a VL CDR1 sequence comprising the amino acid sequence RSSQSLLHSDGYNYLD (SEQ ID NO: 1); a VL CDR2 sequence comprising the amino acid sequence LGSNRAS (SEQ ID NO: 2): and a VL CDR3 sequence comprising the amino acid sequence MQALQTPWT (SEQ ID NO: 3).

In some embodiments, the antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence SYDMH (SEQ ID NO: 7); a VH CDR2 sequence that includes a sequence that is at least 90%, 91%, 92%, 93%, 94%, 95%, 96%, 97%, 98%, 99% or more identical to a VH CDR2 sequence comprising the amino acid sequence VIWYDGSNKYYADSLKG (SEQ ID NO: 8); a VH CDR3 sequence that includes a sequence that is at least 90%, 91%, 92%, 93%, 94%, 95%, 96%, 97%, 98%, 99% or more identical to a VH CDR3 sequence comprising the amino acid sequence VIAARTFYYYGMDV (SEQ ID NO: 9); a VL CDR1 sequence that includes a sequence that is at least 90%, 91%, 92%, 93%, 94%, 95%, 96%, 97%, 98%, 99% or more identical to a VL CDR1 sequence comprising the amino acid sequence comprising the amino acid sequence RSSQSLLHSDGYNYLD (SEQ ID NO: 1); a VL CDR2 sequence that includes a sequence that is at least 90%, 91%, 97%, 93%, 94%, 95%, 96%, 97%, 98%, 99% or more identical to a VL CDR2 sequence comprising the amino acid sequence LGSNRAS (SEQ ID NO: 2); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence MQALQTPWT (SEQ ID NO: 3).

In some embodiments, the antibody or antigen-binding fragment thereof comprises a combination of a VH CDR1 sequence, a VH CDR2 sequence, a VH CDR3 sequence, a VL CDR' sequence, a VL CDR2 sequence, and a VL CDR3 sequence, wherein at least one complementarity determining region (CDR) sequence is selected from the group consisting of a VH CDR1 sequence comprising the amino acid sequence NYWMS (SEQ ID NO: 10); a VH CDR2 sequence comprising the amino acid sequence NIKKDGSEKFYVDSVKG (SEQ ID NO: 11); a VH CDR3 sequence comprising the amino acid sequence EIQLYLQH (SEQ ID NO: 12); a VL CDR1 sequence comprising the amino acid sequence RASQGISNWLA (SEQ ID NO: 4); a VL CDR2 sequence comprising the amino acid sequence AASSLQS (SEQ ID NO: 5); and a VL CDR3 sequence comprising the amino acid sequence QQANSFPLT (SEQ ID NO: 6).

In some embodiments, the antibody or antigen-binding fragment thereof comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence NYWMS (SEQ ID NO: 10); a VH CDR2 sequence that includes a sequence that is at least 90%, 91%, 92%, 93%, 94%, 95%, 96%, 97%, 98%, 99% or more identical to a VH CDR2 sequence comprising the amino acid sequence NIKKDGSEKFYVDSVKG (SEQ ID NO: 11); a VH CDR3 sequence that includes a sequence that is at least 90%, 91%, 92%, 93%, 94%, 95%, 96%, 97%, 98%, 99% or more identical to a VH CDR3 sequence comprising the amino acid sequence EIQLYLQH (SEQ ID NO: 12); a VL CDR1 sequence that includes a sequence that is at least 90%, 91%, 92%, 93%, 94%, 95%, 96%, 97%, 98%, 99% or more identical to a VL CDR1 sequence comprising the amino acid sequence comprising the amino acid sequence RASQGISNWLA (SEQ ID NO: 4); a VL CDR2 sequence that includes a sequence that is at least 90%, 91%, 92%, 93%, 94%, 95%, 96%, 97%, 98%, 99% or more identical to a VL CDR2 sequence comprising the amino acid sequence AASSLQS (SEQ ID NO: 5); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQANSFPLT (SEQ ID NO: 6).

In some embodiments, the antibody or antigen-binding fragment thereof comprises a combination of a variable heavy chain frame work region 1 (VH FR1) sequence, a variable heavy chain frame work region 2 (VH FR2) sequence, a variable heavy chain frame work region 3 (VH FR3) sequence, a variable heavy chain frame work region 4 (VH FR4) sequence; a variable light chain frame work region 1 (VL FR1) sequence, a variable light chain frame work region 2 (VL FR2) sequence, a variable light chain frame work region 3 (VL FR3) sequence, and a variable light chain frame work region 4 (VL FR4) sequence, wherein at least one frame work region (FR) sequence is selected from the group consisting of a VH FR1 sequence comprising the amino acid sequence QVQLVESGGGVVQPGRSLRLSCAASGFTFS (SEQ ID NO: 21); a VH FR2 sequence comprising the amino acid sequence WVRQAPGKGLEWVA (SEQ ID NO: 22); a VH FR3 sequence comprising the amino acid sequence RFTISRDNSKNTLYLQMNSLRAEDTAVYYCAR (SEQ ID NO: 23); a VH FR4 sequence comprising the amino acid sequence WGQGTTVTVSS (SEQ ID NO: 24); a VL FR1 sequence comprising the amino acid sequence DIVMTQSPLSLPVTPGEPASISC (SEQ ID NO: 13); a VL FR2 sequence comprising the amino acid sequence WYLQKSGQSPQLLIY (SEQ ID NO: 14); a VL FR3 sequence comprising the amino acid sequence GVPDRFSGSGSGTDFTLKISRVEAEDVGVYYC (SEQ ID NO: 15); and a VL FR4 sequence comprising the amino acid sequence FGQGTKVEIKR (SEQ ID NO: 16).

In some embodiments, the antibody or antigen-binding; fragment thereof comprises a combination of a variable heavy chain frame work region 1 (VH FR1) sequence, a variable heavy chain frame work region 2 (VH FR2) sequence, a variable heavy chain frame work region 3 (VH FR3) sequence, a variable heavy chain frame work region 4 (VH FR4) sequence, a variable light chain frame work region 1 (VL FR1) sequence, a variable light chain frame work region 2 (VL FR2) sequence, a variable light chain frame work region 3 (VL FR3) sequence, and a variable light chain frame work region 4 (VL FR4) sequence, wherein at least one frame work region (FR) sequence is selected from the group consisting of a VH FR1 sequence comprising the amino acid sequence EVQLVESGGGLVQPGGSLRLSCAASGITFS (SEQ ID NO: 25); a VH FR2 sequence comprising the amino acid sequence WVRQAPGKGLEWVA (SEQ ID NO: 26); a VH FR3 sequence comprising the amino acid sequence RFTISRDNAKNSLYLQINSLRAEDTAMYYCAR (SEQ ID NO: 27); a VH FR4 sequence comprising the amino acid sequence WGQGTLVTVSS (SEQ ID NO: 28); a VL FR1 sequence comprising the amino acid sequence DIQMTQSPSSVSASVGGRVTITC (SEQ ID NO: 17); a VL FR2 sequence comprising the amino acid sequence WYQQKPGKAPKLLIY (SEQ ID NO: 18); a VL FR3 sequence comprising the amino acid sequence GVPSRFSGSGSGTDFTLTISNLQPEDFASYYC (SEQ ID NO: 19); and a VL FR4 sequence comprising the amino acid sequence FGGGTKVEIKR (SEQ ID NO: 20).

In some embodiments, the antibody or antigen-binding fragment thereof is encoded by a nucleic acid sequence that comprises a nucleic acid sequence encoding a heavy chain amino acid sequence comprising SEQ ID NO: 31 or SEQ ID NO: 32. In some embodiments, the antibody or antigen-binding fragment thereof is encoded by a nucleic acid sequence that comprises a nucleic acid sequence encoding a heavy chain amino acid sequence comprising the amino acid sequence selected SEQ ID NO: 31. In some embodiments, the antibody or antigen-binding fragment thereof is encoded by a nucleic acid sequence that comprises a nucleic acid sequence encoding a heavy chain amino acid sequence comprising the amino acid sequence selected SEQ ID NO: 32.

In some embodiments, the antibody or antigen-binding fragment thereof is encoded by a nucleic acid sequence that comprises a nucleic acid sequence encoding a light chain amino acid sequence comprising SEQ ID NO: 29 or SEQ ID NO: 30. In some embodiments, the antibody or antigen-binding fragment thereof is encoded by a nucleic acid sequence that comprises a nucleic acid sequence encoding a light chain amino acid sequence comprising the amino acid sequence selected SEQ ID NO: 29. In some embodiments, the antibody or antigen-binding fragment thereof is encoded by a nucleic acid sequence that comprises a nucleic acid sequence encoding a light chain amino acid sequence comprising the amino acid sequence selected SEQ ID NO: 30.

In some embodiments, the antibody or antigen-binding; fragment thereof is encoded by a nucleic acid sequence that comprises a nucleic acid sequence encoding a heavy chain amino acid sequence comprising the amino acid sequence of SEQ ID NO: 31, and a nucleic acid sequence encoding a light chain amino acid sequence comprising the amino acid sequence SEQ ID NO: 29. In sonic embodiments, the antibody or antigen-binding fragment thereof is encoded by a nucleic acid sequence that comprises a nucleic acid sequence encoding a heavy chain amino acid sequence comprising the amino acid sequence of SEQ ID NO: 32, and a nucleic acid sequence encoding a light chain amino acid sequence comprising the amino acid sequence SEQ ID NO: 30.

In some embodiments, the antibody or antigen-binding fragment thereof is encoded by a nucleic acid sequence that comprises a nucleic acid sequence that is at least 90%, 91%, 92%, 93%, 94?, 95%, 96%, 97%, 98% or 99% identical to a nucleic acid sequence encoding a heavy chain amino acid sequence comprising an amino acid sequence selected from the group consisting of SEQ ID NO: 31 or SEQ ID NO: 32. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising the amino acid sequence of SEQ ID NO: 31. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising the amino acid sequence of SEQ ID NO: 32.

In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light chain amino acid sequence comprising an amino acid sequence selected from the group consisting of SEQ ID NO: 29 or SEQ NO: 30. In some embodiments, the antibody or antigen-binding fragment thereof is encoded by a nucleic acid sequence that comprises a. nucleic acid sequence that is at least 90%, 91%, 92%, 93%, 94%, 95%, 96%, 97%, 98% or 99% identical to a nucleic acid sequence encoding a light chain amino acid sequence comprising the amino acid sequence of SEQ ID NO: 29. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a light chain amino acid sequence comprising the amino acid sequence of SEQ ID NO: 30.

In some embodiments, the antibody or antigen-binding fragment thereof is encoded by a nucleic acid sequence that comprises a nucleic acid sequence that is at least 90%, 91%, 97%, 93%, 94%, 95%, 96%, 97%, 98% or 99% identical to a nucleic acid sequence encoding a heavy chain amino acid sequence comprising the amino acid sequence of SEQ ID NO: 31, and a nucleic acid sequence that is at least 90%, 91%, 92%, 93%, 94%, 95%, 96%, 97%, 98% or 99% identical to a nucleic add sequence encoding a light chain amino acid sequence comprising the amino acid sequence of SEQ ID NO: 29. In some embodiments, the antibody or antigen-binding fragment thereof is encoded by a nucleic acid sequence that comprises a nucleic acid sequence that is at least 90%, 91%, 92%, 93%, 94%, 95%, 96%, 97%, 98% or 99% identical to a nucleic acid sequence encoding a heavy chain amino acid sequence comprising the amino acid sequence of SEQ ID NO: 32, and a nucleic acid sequence that is at least 90%, 91%, 92%, 93%, 94%, 95%, 96%, 97%, 98% or 99% identical to a nucleic acid sequence encoding a light chain amino acid sequence comprising the amino acid sequence of SEQ ID NO: 30.

In some embodiments, the antibody or antigen-binding fragment thereof is encoded by a nucleic acid sequence encoding a heavy chain amino acid sequence comprising the nucleic acid sequence SEQ ID NO: 35 or SEQ ID NO: 36. In some embodiments, the antibody or antigen-binding fragment thereof is encoded by a nucleic acid sequence encoding a heavy chain amino acid sequence comprising the nucleic acid sequence SEQ ID NO: 35. In some embodiments, the antibody or antigen-binding fragment thereof is encoded by a nucleic acid sequence encoding a heavy chain amino acid sequence comprising the nucleic acid sequence selected SEQ ID NO: 36.

In some embodiments, the antibody or antigen-binding fragment thereof is encoded by a nucleic acid sequence encoding a. light chain amino add sequence comprising the nucleic acid sequence SEQ ID NO: 33 or SEQ ID NO: 34. In some embodiments, the antibody or antigen-binding fragment thereof is encoded by a nucleic acid sequence encoding a light chain amino acid sequence comprising the nucleic acid sequence SEQ ID NO: 33. In some embodiments, the antibody or antigen-binding fragment thereof is encoded by a nucleic acid sequence encoding a light chain amino acid sequence comprising the nucleic acid sequence selected SEQ ID NO: 34.

In some embodiments, the antibody or antigen-binding fragment thereof is encoded by a nucleic acid sequence encoding a heavy chain amino acid sequence comprising the nucleic acid sequence of SEQ ID NO: 35, and a nucleic acid sequence encoding a light chain amino acid sequence comprising the nucleic acid sequence SEQ ID NO: 33. In some embodiments, the antibody or antigen-binding fragment thereof is encoded by a nucleic acid sequence encoding a heavy chain amino acid sequence comprising the nucleic acid sequence of SEQ ID NO: 36, and a nucleic acid sequence encoding a light chain amino acid sequence comprising the nucleic acid sequence SEQ ID NO: 34.

In some embodiments, the antibody or antigen-binding fragment thereof is incorporated in a multispecific antibody or antigen-binding fragment thereof, where at least one arm of the multispecific antibody or antigen-binding fragment thereof specifically binds PSMA. In some embodiments, the antibody or antigen-binding fragment thereof is incorporated in a bispecific antibody or antigen-binding fragment thereof, where at least one arm of the bispecific antibody or antigen-binding fragment thereof specifically binds PSMA.

In some embodiments, at least one arm of the multi specific antibody or antigen-binding fragment thereof, e.g., a bispecific antibody or antigen-binding fragment thereof, comprises a heavy chain variable region amino acid sequence comprising an amino acid sequence selected from the group consisting of SEQ ID NO: 31 or SEQ ID NO: 32. In some embodiments, at least one arm of the multispecific antibody or antigen-binding fragment thereof, e.g., a bispecific antibody or antigen-binding fragment thereof, comprises a heavy chain variable region amino acid sequence comprising the amino acid sequence of SEQ ID NO: 31. In some embodiments, at least one arm of the multispecific antibody or antigen-binding fragment thereof, e.g., a bispecific antibody or antigen-binding fragment thereof, comprises a heavy chain variable region amino acid sequence comprising the amino acid sequence of SEQ ID NO: 32.

In some embodiments, at least one arm of the multi specific antibody or antigen-binding fragment thereof, e.g., a bispecific antibody or antigen-binding fragment thereof, comprises a light chain variable region amino acid sequence comprising an amino acid sequence selected from the group consisting of SEQ ID NO: 29 or SEQ ID NO: 30. In some embodiments, at least one arm of the multispecific antibody or antigen-binding fragment thereof, e.g., a bispecific antibody or antigen-binding fragment thereof, comprises a light chain variable region amino acid sequence comprising an amino acid sequence of SEQ ID NO: 29. In some embodiments, at least one arm of the multispecific antibody or antigen-binding fragment thereof, e.g., a bispecific antibody or antigen-binding fragment thereof, comprises a light chain variable region amino acid sequence comprising the amino acid sequence of SEQ ID NO: 30.

In some embodiments, at least one arm of the multi specific antibody or antigen-binding fragment thereof, e.g., a bispecific antibody or antigen-binding fragment thereof, comprises a heavy chain variable region amino acid sequence comprising an amino acid sequence selected from the group consisting of SEQ ID NO: 31 and SEQ ID NO: 32, and a light chain variable region amino acid sequence comprising an amino acid sequence selected from the group consisting of SEQ ID NO: 29 and SEQ ID NO: 30.

In some embodiments, at least one arm of the multispecific antibody or antigen-binding fragment thereof, e.g., a bispecific antibody or antigen-binding fragment thereof, comprises a heavy chain variable region amino acid sequence comprising an amino acid sequence selected from the group consisting of SEQ ID NO: 31 and SEQ ID NO: 32, and a light chain variable region amino acid sequence comprising an amino acid sequence selected from the group consisting of SEQ ID NO: 29 and SEQ ID NO: 30. In some embodiments, at least one arm of the multispecific antibody or antigen-binding fragment thereof, e.g., a bispecific antibody or antigen-binding fragment thereof, comprises a heavy chain variable region amino acid sequence comprising the amino acid sequence of SEQ ID NO: SEQ ID NO: 31, and a light chain variable region amino acid sequence comprising the amino acid sequence of SEQ ID NO: 29, a bispecific antibody or antigen-binding fragment thereof, comprises a heavy chain variable region amino acid sequence comprising the amino acid sequence of SEQ ID NO: SEQ ID NO: 32, and a light chain variable region amino acid sequence comprising the amino acid sequence of SEQ ID NO: 30.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence comprising an amino acid sequence selected from the group consisting of SEQ ID NO: 31 and SEQ ID NO: 32. In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence comprising the amino acid sequence of SEQ NO: 31. In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence comprising the amino acid sequence of SEQ ID NO: 32.

In some embodiments, at least one arm of the multispecific antibody or antigen-binding fragment thereof, e.g., a bispecific antibody or antigen-binding fragment thereof, comprises a light chain variable region amino acid sequence that is at least 90%, 91%, 92%, 93%, 94%, 95%, 96%, 97%, 98% or 99% identical to an amino acid sequence comprising an amino acid sequence selected from the group consisting of SEQ ID NO: 29 and SEQ ID NO: 30. In some embodiments, at least one arm of the multispecific antibody or antigen-binding fragment thereof, e.g., a bispecific antibody or antigen-binding fragment thereof, comprises a light chain variable region amino acid sequence that is at least 90%, 91%, 92%, 93%, 94%, 95%, 96%, 97%, 98% or 99% identical to an amino acid sequence comprising an amino acid sequence of SEQ NO: 29. In some embodiments, at least one arm of the multispecific antibody or antigen-binding fragment thereof, e.g., a bispecific antibody or antigen-binding fragment thereof, comprises a light chain variable region amino acid sequence that is at least 90%, 91%, 92%, 93%, 94%, 95%, 96%, 97%, 98% or 99% identical to an amino acid sequence comprising the amino acid sequence of SEQ ID NO: 30.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence of SEQ ID NO: 31, and a light chain variable region amino acid sequence that is at least 90%, 91%, 92%, 93%, 94%, 95%, 96%, 97%, 98% or 99% identical to an amino acid sequence comprising of SEQ ID NO: 29.

In some embodiments, at least one arm of the multispecific antibody or antigen-binding fragment thereof, e.g., a bispecific antibody or antigen-binding fragment thereof, comprises a heavy chain variable region amino acid sequence that is at least 90%, 91%, 92%, 93%, 94%, 95%, 96%, 97%, 98% or 99% identical to an amino acid sequence of SEQ ID NO: 32, and a light chain variable region amino acid sequence that is at least 90%, 91%, 92%, 93%, 94%, 95%, 96%, 97%, 98% or 99% identical to an amino acid sequence comprising of SEQ ID NO: 30.

In some embodiments, at least one arm of the multispecific antibody or antigen-binding fragment thereof, e.g., a bispecific antibody or antigen-binding fragment thereof, comprises a combination of a variable heavy chain complementarity determining region 1 (VH CDR1, also referred to herein as CDRH1) sequence, a variable heavy chain complementarity determining region 2 (VH CDR2, also referred to herein as CDRH2) sequence, a variable heavy chain complementarity determining region 3 (VH CDR3, also referred to herein as CDRH3) sequence, a variable light chain complementarity determining region 1 (VL CDR1, also referred to herein as CDRL1) sequence, a variable light chain complementarity determining region 2 (VL CDR2, also referred to herein as CDRL2) sequence, and a variable light chain complementarity determining region 3 (VL CDR3, also referred to herein as CDRL3) sequence, wherein at least one complementarity determining region (CDR) sequence is selected from the group consisting of a VH CDR1 sequence comprising the amino acid sequence SYDMH (SEQ ID NO: 7) or NYWMS (SEQ ID NO: 10); a VH CDR2 sequence comprising the amino acid sequence VIWYDGSNKYYADSLKG (SEQ ID NO: 8) or NIKKDGSEKFYVDSVKG (SEQ ID NO: 11); a VH CDR3 sequence comprising the amino acid sequence VIAARTFYYYGMDV (SEQ ID NO: 9) or EIQLYLQH (SEQ ID NO: 12); a VL CDR1 sequence comprising the amino acid sequence RSSQSLLHSDGYNYLD (SEQ ID NO: 1) or RASQGISNWLA (SEQ ID NO: 4); a VL CDR2 sequence comprising the amino acid sequence LGSNRAS (SEQ NO: 2) or AASSLQS (SEQ ID NO: 5); and a VL CDR3 sequence comprising the amino acid sequence MQALQTPWT (SEQ ID NO: 3) or QQANSFPLT (SEQ ID NO: 6).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at one complementarity determining region (CDR) sequence is selected from the group consisting of a VH CDR1 sequence comprising the amino acid sequence SYDMH (SEQ ID NO: 7); a VH CDR2 sequence comprising the amino acid sequence VIWYDGSNKYYADSLKG (SEQ ID NO: 8); a VH CDR3 sequence comprising the amino acid sequence VIAARTFYYYGMDV (SEQ ID NO: 9); a VL CDR1 sequence comprising the amino acid sequence RSSQSLLHSDGYNYLD (SEQ ID NO: 1); a VL CDR2 sequence comprising the amino acid sequence LGSNRAS (SEQ ID NO: 2); and a VL CDR3 sequence comprising the amino acid sequence MQALQTPWT (SEQ ID NO: 3).

In some embodiments, at least one arm of the multi specific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1, sequence that includes a sequence that is at least 90%, 91%, 92%, 93%, 94%, 95%, 96%, 97%, 98%, 99% or more identical to a VH CDR1 sequence comprising the amino acid sequence SYDMH (SEQ ID NO: 7); a VH CDR2 sequence that includes a sequence that is at least 90%, 91%, 92%, 93%, 94%, 95%, 96%, 97%, 98%, 99% or more identical to a VH CDR2 sequence comprising the amino acid sequence VIWYDGSNKYYADSLKG (SEQ ID NO: 8); a VH CDR3 sequence that includes a sequence that is at least 90%, 91%, 92%, 93%, 94%, 95%, 96%, 97%, 98%, 99% or more identical to a VH CDR3 sequence comprising the amino acid sequence VIAARTFYYYGMDV (SEQ ID NO: 9); a VL CDR1 sequence that includes a sequence that is at least 90%, 91%, 92%, 93%, 94%, 95%, 96%, 97%, 98%, 99% or more identical to a VL CDR1 sequence comprising the amino acid sequence comprising the amino acid sequence RSSQSLLHSDGYNYLD (SEQ ID NO: 1); a VL CDR2 sequence that includes a sequence that is at least 90%, 91%, 92%, 93%, 94%, 95%, 96%, 97%, 98%, 99% or more identical to a VL CDR2 sequence comprising the amino acid sequence LGSNRAS (SEQ ID NO: 2); and a VL CDR3 sequence that includes a sequence that is at least 90%, 91%, 97%, 93%, 94%, 95%, 96%, 97%, 98%, 99% or more identical to a VL CDR3 sequence comprising the amino acid sequence MQALQTPWT (SEQ ED NO: 3).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at least one complementarity determining region (CDR) sequence is selected from the group consisting of a CDR1 sequence comprising the amino acid sequence NYWMS (SEQ ID NO: 10); a VH CDR2 sequence comprising the amino acid sequence NIKKDGSEKFYVDSVKG (SEQ ID NO: 11); a VH CDR3 sequence comprising the amino acid sequence EIQLYLQH (SEQ ID NO: 12); a VL CDR1 sequence comprising the amino acid sequence RASQGISNWLA (SEQ ID NO: 4); a VL CDR2 sequence comprising the amino acid sequence AASSLQS (SEQ ID NO: 5); and a VL CDR3 sequence comprising the amino acid sequence QQANSFPLT (SEQ ID NO: 6).

In some embodiments, at least one arm of the multispecific antibody or antigen-binding fragment thereof, e.g., a bispecific antibody or antigen-binding fragment thereof, comprises a combination of a VH CDR1 sequence, a VH CDR2 sequence, a VH CDR3 sequence, a VL CDR1 sequence, a VL CDR2 sequence, and a VL CDR3 sequence, wherein at least one CDR sequence is selected from the group consisting of a VH CDR1 sequence that includes a sequence that is at least 90%, 91%, 92%, 93%, 94%, 95%, 96%, 97%, 98%, 99% or more identical to a VH CDR1 sequence comprising the amino acid sequence NYWMS (SEQ ID NO: 10); a VH CDR2 sequence that includes a sequence that is at least 90%, 91%, 92%, 93%, 94%, 95%, 96%, 97%, 98%, 99% or more identical to a VH CDR2 sequence comprising the amino acid sequence NIKKDGSEKFYVDSVKG (SEQ ID NO: 11); a VH CDR3 sequence that includes a sequence that is at least 90%, 91%, 92%, 93%, 94%, 95%, 96%, 97%, 98%, 99% or more identical to a VH CDR3 sequence comprising the amino acid sequence EIQLYLQH (SEQ ID NO: 12); a VL CDR1 sequence that includes a sequence that is at least 90%, 91%, 92%, 93%, 94%, 95%, 96%, 97%, 98%, 99% or more identical to a VL CDR1 sequence comprising the amino acid sequence comprising the amino acid sequence RASQGISNWLA (SEQ ID NO: 4); a VL CDR2 sequence that includes a sequence that is at least 90%, 91%, 92%, 93%, 94%, 95%, 96%, 97%, 98%, 99% or more identical to a VL CDR2 sequence comprising the amino acid sequence AASSLQS (SEQ ID NO: 5); and a VL CDR3 sequence that includes a sequence that is at least 90%, 91%, 92%, 93%, 94%, 95%, 96%, 97%, 98%, 99% or more identical to a VL CDR3 sequence comprising the amino acid sequence QQANSFPLT (SEQ ID NO: 6).

Suitable anti-PSMA antibodies of the disclosure also include an antibody or antigen binding fragment thereof that binds to the same epitope on human PSMA and/or cynomolgus monkey PSMA as an anti-PSMA antibody comprising a heavy chain variable region amino acid sequence selected from the group consisting of SEQ ID NO: 31 and SEQ ID NO: 32, and a light chain variable region amino acid sequence selected from the group consisting of SEQ ID NO: 29 and SEQ ID NO: 30.

Suitable anti-PSMA antibodies of the disclosure also include an antibody or antigen binding fragment thereof that binds to the same epitope on human PSMA and/or cynomolgus monkey PSMA as an anti-PSMA antibody comprises the VH CDR1 sequence comprising the amino acid sequence SYDMH (SEQ ID NO: 7); the VH CDR2 sequence comprising the amino acid sequence VIWYDGSNKYYADSLKG (SEQ ID NO: 8); the VH CDR3 sequence comprising the amino acid sequence VIAARTFYYYGMDV (SEQ ID NO: 9); the VL CDR1 sequence comprising the amino acid sequence RSSQSLLHSDGYNYLD (SEQ ID NO: 1); the VL CDR2 sequence comprising the amino acid sequence LGSNRAS (SEQ ID NO: 2); and the VL CDR3 sequence comprising the amino acid sequence MQALQTPWT (SEQ ID NO: 3).

Suitable anti-PSMA antibodies of the disclosure also include an antibody or antigen binding fragment thereof that binds to the same epitope on human PSMA and/or cynomolgus monkey PSMA as an anti-PSMA antibody comprises the VH CDR1 sequence comprising the amino acid sequence NYWMS (SEQ ID NO: 10); the VH CDR2 sequence comprising the amino acid sequence NIKKDGSEKFYVDSVKG (SEQ ID NO: 11); the VH CDR3 sequence comprising the amino acid sequence EIQLYLQH (SEQ ID NO: 12); the VL CDR1 sequence comprising the amino acid sequence RASQGISNWLA (SEQ ID NO: 4); the VL CDR2 sequence comprising the amino acid sequence AASSLQS (SEQ ID NO: 5); and the VL CDR3 sequence comprising the amino acid sequence QQANSFPLT (SEQ ID NO: 6).

Suitable anti-PSMA antibodies of the disclosure also include an antibody or antigen binding fragment thereof that cross-competes for binding to human PSMA and/or cynomolgus monkey PSMA as an anti-PSMA antibody comprising a heavy chain variable region amino acid sequence selected from the group consisting of SEQ ID NO: 31 and SEQ ID NO: 32, and a light chain variable region amino acid sequence selected from the group consisting of SEQ ID NO: 29 and SEQ ID NO: 30.

Suitable anti-PSMA antibodies of the disclosure also include an antibody or antigen binding fragment thereof that cross-competes for binding to human PSMA and/or cynomolgus monkey PSMA as an anti-PSMA antibody comprises the VH CDR1 sequence comprising the amino acid sequence SYDMH (SEQ ID NO: 7); the VH CDR2 sequence comprising the amino acid sequence VIWYDGSNKYYADSLKG (SEQ ID NO: 8); the VH CDR3 sequence comprising the amino acid sequence VIAARTFYYYGMDV (SEQ ID NO: 9); the VL CDR1 sequence comprising the amino acid sequence RSSQSLLHSDGYNYLD (SEQ ID NO: 1); the VL CDR2 sequence comprising the amino acid sequence LGSNRAS (SEQ ID NO: 2); and the VL CDR3 sequence comprising the amino acid sequence MQALQTPWT (SEQ ID NO: 3).

Suitable anti-PSMA antibodies of the disclosure also include an antibody or antigen binding fragment thereof that cross-competes for binding to human PSMA and/or cynomolgus monkey PSMA as an anti-PSMA antibody comprises the VH CDR1 sequence comprising the amino acid sequence NYWMS (SEQ ID NO: 10); the VH CDR2 sequence comprising the amino acid sequence NIKKDGSEKFYVDSVKG (SEQ ID NO: 11); the VH CDR3 sequence comprising the amino acid sequence EIQLYLQH (SEQ ID NO: 12); the VL CDR1 sequence comprising the amino acid sequence RASQGISNWLA (SEQ ID NO: 4); the VL CDR2 sequence comprising the amino acid sequence AASSLQS (SEQ ID NO: 5); and the VL CDR3 sequence comprising the amino acid sequence QQANSFPLT (SEQ ID NO: 6).

The invention also provides methods of treating, preventing and/or delaying the onset or progression of, or alleviating a symptom associated with aberrant expression and/or activity of PSMA in a subject using activatable antibodies that bind PSMA, particularly activatable antibodies that bind and neutralize or otherwise inhibit at least one biological activity of PSMA and/or PSMA-mediated signaling.

The invention also provides methods of treating, preventing and/or delaying the onset or progression of, or alleviating a symptom associated with the presence, growth, proliferation, metastasis, and/or activity of cells which are expressing PSMA or aberrantly expressing PSMA in a subject that bind, target, neutralize, kill, or otherwise inhibit at least one biological activity of cells which are expressing or aberrantly expressing PSMA.

The invention also provides methods of treating, preventing and/or delaying the onset or progression of, or alleviating a symptom associated with the presence, growth, proliferation, metastasis, and/or activity of cells which are expressing PSMA in a subject that bind, target, neutralize, kill, or otherwise inhibit at least one biological activity of cells which are expressing PSMA.

The invention also provides methods of treating, preventing and/or delaying the onset or progression of, or alleviating a symptom associated with the presence, growth, proliferation, metastasis, and/or activity of cells which are aberrantly expressing PSMA in a subject that bind, target, neutralize, kill, or otherwise inhibit at least one biological activity of cells which are aberrantly expressing PSMA.

In some embodiments, the mammalian PSMA is selected from the group consisting of a human PSMA and a cynomolgus monkey PSMA. In some embodiments, the AB specifically binds to human PSMA or cynomolgus monkey PSMA with a dissociation constant of less than 1 nM. In some embodiments, the mammalian PSMA is a human PSMA. In some embodiments, the mammalian PSMA is a cynomolgus PSMA.

In some embodiments, the AB has one or more of the following characteristics: (a) the AB specifically binds to human PSMA; and (b) the AB specifically binds to human PSMA and cynomolgus monkey PSMA.

In some embodiments, the AB blocks the ability of a natural ligand or receptor to bind to the mammalian PSMA with an EC50 less than or equal to 5 nM, less than or equal to 10 nM, less than or equal to 50 nM, less than or equal to 100 nM, less than or equal to 500 nM, and/or less than or equal to 1000 nM.

In some embodiments, the AB blocks the ability of a natural ligand to bind to the mammalian PSMA with an EC50 of 5 nM to 1000 nM, 5 nM to 500 nM, 5 nM to 100 nM 5 nM to 50 nM, 5 nM to 10 nM, 10 nM to 1000 nM, 10 nM to 500 nM, 10 nM to 100 nM 10 nM to 50 nM, 50 nM to 1000 nM, 50 nM to 500 nM, 50 nM to 100 nM, 100 nM to 1000 nM, 100 nM to 500 nM, 500 nM to 1000 nM.

In some embodiments, the AB of the present disclosure inhibits or reduces the growth, proliferation, and/or metastasis of cells expressing mammalian PSMA. Without intending to be bound by any theory, the AB of the present disclosure may inhibit or reduce the growth, proliferation, and/or metastasis of cells expressing mammalian PSMA by specifically binding to PSMA and inhibiting, blocking, and/or preventing the binding of a natural ligand or receptor to mammalian PSMA.

In some embodiments, the antibody includes an agent conjugated to the AB. In some embodiments, the agent conjugated to the AB or the AB of an antibody is a therapeutic agent. In some embodiments, the agent is an antineoplastic agent. In some embodiments, the agent is a toxin or fragment thereof. As used herein, a fragment of a toxin is a fragment that retains toxic activity. In some embodiments, the agent is conjugated to the AB via a cleavable linker. In some embodiments, the agent is conjugated to the AB via a noncleavable linker. In some embodiments, the agent is conjugated to the AB via a linker that is cleavable in an intracellular or lysosomal environment. In some embodiments, the agent is a microtubule inhibitor. In some embodiments, the agent is a nucleic acid damaging agent, such as a DNA alkylator, a DNA cleaving agent, a DNA cross-linker, a DNA intercalator, or other DNA damaging agent. In some embodiments, the agent is an agent selected from the group listed in Table 5. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine. In some embodiments, the agent is a pyrrolobenzodiazepine dimer.

In some embodiments, the agent comprises a molecule having a structure of formula (I):

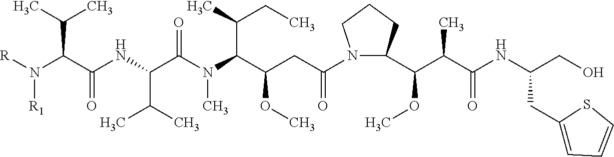

wherein R1 is a hydrogen or a C₁₋₆ alkyl group; wherein R is selected from the group consisting of: a hydrogen, a C₁₋₆ alkyl, a linker, or a group X1 -Y1-* wherein * is the point of attachment to the nitrogen; and wherein Y1 is an oxycarbonyl group and X1 is a C₁₋₆ alkyl group, a 9-fluorenylmethyl group, a benzyl group, or a tert-butyl group In some embodiments, the antibody is conjugated to one or more equivalents of an agent.

In some embodiments, the agent is conjugated to the antibody via a linker having a structure of formula (II):

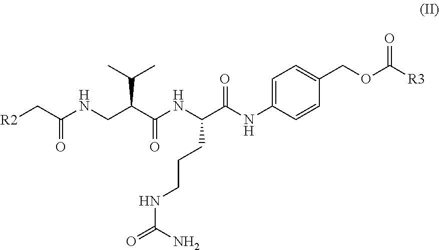

wherein R3 is an agent attached to formula (II) where the point of attachment is a nitrogen, sulfur, oxygen, or carbon atom; and wherein R2 is a moiety attached to formula (II) wherein the point of attachment is selected from the group consisting of: a chlorine group, an iodine group, a bromine group, and a thiol group.

In some embodiments, the agent is conjugated to the antibody via a linker, wherein the agent and linker has a structure of formula (III):

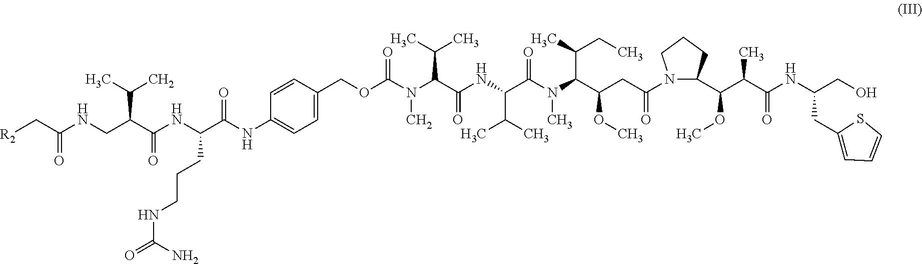

wherein R2 is a point of attachment to the AB.

In some embodiments, the antibody is conjugated to one equivalent of the agent. In some embodiments, the antibody is conjugated to two, three, four, five, six, seven, eight, nine, ten, or greater than ten equivalents of the agent. In some embodiments, the antibody is part of a mixture of antibodies having a homogeneous number of equivalents of conjugated agents. In some embodiments, the antibody is part of a mixture of antibodies having a heterogeneous number of equivalents of conjugated agents. In some embodiments, the mixture of antibodies is such that the average number of agents conjugated to each antibody is between zero to one, between one to two, between two and three, between three and four, between four and five, between five and six, between six and seven, between seven and eight, between eight and nine, between nine and ten, and ten and greater. In some embodiments, the mixture of antibodies is such that the average number of agents conjugated to each antibody is one, two, three, four, five, six, seven, eight, nine, ten, or greater. In some embodiments, the antibody comprises one or more site-specific amino acid sequence modifications such that the number of lysine and/or cysteine residues is increased or decreased with respect to the original amino acid sequence of the antibody, thus in some embodiments correspondingly increasing or decreasing the number of agents that can be conjugated to the antibody, or in some embodiments limiting the conjugation of the agents to the antibody in a site-specific manner. In some embodiments, the modified antibody is modified with one or more non-natural amino acids in a site-specific manner, thus in some embodiments limiting the conjugation of the agents to only the sites of the non-natural amino acids.

In some embodiments, the agent is an anti-inflammatory agent. In some embodiments, the antibody also includes a detectable moiety. In some embodiments, the detectable moiety is a diagnostic agent.

In some embodiments, the AB of the antibody naturally contains one or more disulfide bonds. In some embodiments, the AB can be engineered to include one or more disulfide bonds.

In some embodiments, the antibody drug conjugates (ADCs) can include one or more polypeptides that include the combination of a light chain sequence or a light chain variable domain sequence, and a heavy chain sequence or a heavy chain variable domain sequences, a linker, and a toxin.

In some embodiments, the anti-PSMA antibody, or anti-PSMA conjugated antibody is administered during and/or after treatment in combination with one or more additional agents such as, for example, a chemotherapeutic agent, an anti-inflammatory agent, and/or an immunosuppressive agent. In some embodiments, the anti-PSMA antibody or conjugated anti-PSMA antibody, and the additional agent are formulated into a single therapeutic composition, and the anti-PSMA antibody or conjugated anti-PSMA antibody, and additional agent are administered simultaneously. Alternatively, the anti-PSMA antibody or conjugated anti-PSMA antibody, and additional agent are separate from each other, e.g., each is formulated into a separate therapeutic composition, and the anti-PSMA antibody or conjugated anti-PSMA antibody, and the additional agent are administered simultaneously, or the anti-PSMA antibody or conjugated anti-PSMA antibody, and the additional agent are administered at different times during a treatment regimen. For example, the anti-PSMA antibody or conjugated anti-PSMA antibody, is administered prior to the administration of the additional agent, the anti-PSMA antibody or conjugated anti-PSMA antibody, is administered subsequent to the administration of the additional agent, or the anti-PSMA antibody or conjugated anti-PSMA antibody, and the additional agent are administered in an alternating fashion. As described herein, the anti-PSMA antibody or conjugated anti-PSMA antibody, and additional agent are administered in single doses or in multiple doses.

In some embodiments, the anti-PSMA antibody or conjugated anti-PSMA. antibody, and the additional agent(s) are administered simultaneously. For example, the anti-PSMA antibody or conjugated anti-PSMA antibody, and the additional agent(s) can be formulated in a single composition or administered as two or more separate compositions. In some embodiments, the anti-PSMA antibody or conjugated anti-PSMA antibody, and the additional agent(s) are administered sequentially, or the anti-PSMA antibody or conjugated anti-PSMA antibody, and the additional agent are administered at different times during a treatment regimen.

In some embodiments, the anti-PSMA antibody or conjugated anti-PSMA antibody, is administered during and/or after treatment in combination with one or more additional agents such as, by way of non-limiting example, a chemotherapeutic agent, an anti-inflammatory agent, and/or an immunosuppressive agent, such as an alkylating agent, an anti-metabolite, an anti-microtubule agent, a topoisomerase inhibitor, a cytotoxic antibiotic, and/or any other nucleic acid damaging agent. In some embodiments, the additional agent is a taxane, such as paclitaxel (e.g., Abraxane®). In some embodiments, the additional agent is an anti-metabolite, such as gemcitabine. In some embodiments, the additional agent is an alkylating agent, such as platinum-based chemotherapy, such as carboplatin or cisplatin. In some embodiments, the additional agent is a targeted agent, such as a kinase inhibitor, e.g., sorafenib or erlotinib. In some embodiments, the additional agent is a targeted agent, such as another antibody, e.g., a monoclonal antibody (e.g., bevacizumab), a bispecific antibody, or a multispecific antibody. In some embodiments, the additional agent is a proteosome inhibitor, such as bortezomib or carfilzomib. In some embodiments, the additional agent is an immune modulating agent, such as lenolidominde or IL-2. In some embodiments, the additional agent is radiation. In some embodiments, the additional agent is an agent considered standard of care by those skilled in the art. In some embodiments, the additional agent is a chemotherapeutic agent well known to those skilled in the art.

In some embodiments, the additional agent is another antibody or antigen-binding fragment thereof another conjugated antibody or antigen-binding fragment thereof, another activatable antibody or antigen-binding fragment thereof and/or another conjugated activatable antibody or antigen-binding fragment thereof. In some embodiments the additional agent is another antibody or antigen-binding fragment thereof, another conjugated antibody or antigen-binding fragment thereof, another activatable antibody or antigen-binding fragment thereof and/or another conjugated activatable antibody or antigen-binding fragment thereof against the same target as the first antibody or antigen-binding fragment thereof, the first conjugated antibody or antigen-binding fragment thereof, activatable antibody or antigen-binding fragment thereof and/or a conjugated activatable antibody or antigen-binding fragment thereof, e.g., against PSMA. In some embodiments the additional agent is another antibody or antigen-binding fragment thereof, another conjugated antibody or antigen-binding fragment thereof, another activatable antibody or antigen-binding fragment thereof and/or another conjugated activatable antibody or antigen-binding fragment thereof against a target different than the target of the first antibody or antigen-binding fragment thereof, the first conjugated antibody or antigen-binding fragment thereof, activatable antibody or antigen-binding fragment thereof and/or a conjugated activatable antibody or antigen-binding fragment thereof.

In some embodiments, the additional antibody or antigen binding fragment thereof, conjugated antibody or antigen binding fragment thereof, activatable antibody or antigen binding fragment thereof, and/or conjugated activatable antibody or antigen binding fragment thereof is a monoclonal antibody, domain antibody, single chain, Fab fragment, a F(ab′)2 fragment, a scFv, a scAb, a dAb, a single domain heavy chain antibody, or a single domain light chain antibody. In some embodiments, the additional antibody or antigen binding fragment thereof, conjugated antibody or antigen binding fragment thereof, activatable antibody or antigen binding fragment thereof, and/or conjugated activatable antibody or antigen binding fragment thereof is a mouse, other rodent, chimeric, humanized or fully human monoclonal antibody.

The disclosure also provides methods of producing an anti-PSMA antibody polypeptide by culturing a cell under conditions that lead to expression of the polypeptide, wherein the cell comprises an isolated nucleic acid molecule encoding an antibody described herein, and/or vectors that include these isolated nucleic acid sequences. The disclosure provides methods of producing an antibody by culturing a cell under conditions that lead to expression of the antibody, wherein the cell comprises an isolated nucleic acid molecule encoding an antibody described herein, and/or vectors that include these isolated nucleic acid sequences.

The invention also provides a method of manufacturing antibodies that binds PSMA by (a) culturing a cell comprising a nucleic acid construct that encodes the antibody under conditions that lead to expression of the antibody, wherein the antibody or the antigen binding fragment thereof (AB) specifically binds PSMA; and (b) recovering the antibody.

The invention provides methods of preventing, delaying the progression of, treating, alleviating a symptom of, or otherwise ameliorating an PSMA mediated disease in a subject by administering a therapeutically effective amount of an anti-PSMA antibody, and/or conjugated anti-PSMA antibody described herein to a subject in need thereof.

The invention also provides methods of preventing, delaying the progression of treating, alleviating a symptom of, or otherwise ameliorating cancer in a subject by administering a therapeutically effective amount of an anti-PSMA antibody, and/or conjugated anti-PSMA antibody described herein to a subject in need thereof. Prostate-specific membrane antigen (PSMA) is a type 2 transmembrane glycoprotein with high and restricted expression in all forms of prostate tissue, including carcinoma. Studies have consistently demonstrated PSMA expression in all types of prostate tissue and increased PSMA expression in cancer tissue. PSMA is also expressed in other cancers, more specifically in the neovasculature associated with these cancers.

### BRIEF DESCRIPTION OF THE DRAWINGS

FIG. 1 presents the Tms of the two anti-PSMA antibodies: Fab Tm of cHv75-2a11.G1(L328C)k was 81.0° C., and Fab Tin of cHv75-2a7.G1(C99Y;L128C)k was 73.6° C.

FIG. 2 presents the binding affinities of the two anti-PSMA antibodies to hPSMA by FACS.

FIGS. 3a and 3b present the binding affinities of the two anti-PSMA antibodies to cyno and human PSMA by ELISA (FIG. 3a) and FACS (FIG. 3b).

FIG. 4 presents conjugations of the two anti-PSMA antibodies do not affect antibody's binding affinity to PSMA.

FIG. 5 presents PK studies indicating the stability of the two conjugated anti-PSMA antibodies.

FIG. 6a presents exemplary pharmacokinetic properties of ADCs of the present disclosure.

FIG. 6b present in vitro cytotoxicity of the two conjugated anti-PSMA antibodies in MDA Pca 2b cells.

FIGS. 7a and 7b presents in vivo toxicity of the two conjugated anti-PSMA antibodies in a LAPC9AI tumor mouse model. FIG. 7a shows tumor regression after administrating ADCs into the subcutaneous human prostate carcinoma model LAPC9AI in CB17 SCID mice. FIG. 7b shows the minimum dose of ADCs for tumor regression (mg/kg).

FIGS. 8a and 8b presents in vivo toxicity of the two conjugated anti-PSMA antibodies in a LNCaP tumor mouse model. FIG. 7a shows tumor regression after administrating ADCs into the subcutaneous human prostate carcinoma model LNCaP in CB17 SCID mice. FIG. 8b shows the minimum dose of ADCs for tumor regression (mg/kg).

### DETAILED DESCRIPTION OF THE INVENTION

The present disclosure provides monoclonal antibodies (mAbs) and anti-PSMA drug conjugates that specifically bind PSMA.

In some embodiments, a target-binding moiety to which compounds of the present disclosure can be conjugated include anti-PSMA antibodies, examples of which are described in the sequences below:

TABLE 1
VL CDR Amino Acid Sequences

| | VL CDR1 | VL CDR2 | VL CDR3 |
|---|---|---|---|
| | (SEQ ID | (SEQ ID | (SEQ ID |
| Antibody | NO:) | NO:) | NO:) |
| [Ag]AB-4 | SEQUENCE | SEQUENCE | SEQUENCE |
| cHv75- | (SEQ ID | (SEQ ID | (SEQ ID |
| 2a11.G1 | NO: 1) | NO: 2) | NO: 3) |
| (L328C)k | RSSQSLLH | LGSNRAS | MQALQTPWT |
| | SDGYNYLD | | |
| [Ag]AB-5 | SEQUENCE | SEQUENCE | SEQUENCE |
| cHv75- | (SEQ ID | (SEQ ID | (SEQ ID |
| 2a7.G1(C99Y; | NO: 4) | NO: 5) | NO: 6) |
| L328C)k | RASQGISNWLA | AASSLQS | QQANSFPLT |

TABLE 2
VH CDR Amino Acid Sequences

| | VL CDR1 | VL CDR2 | VL CDR3 |
|---|---|---|---|
| | (SEQ ID | (SEQ ID | (SEQ ID |
| Antibody | NO:) | NO:) | NO:) |
| [Ag]AB-4 | SEQUENCE | SEQUENCE | SEQUENCE |
| cHv75- | (SEQ ID | (SEQ ID | (SEQ ID |
| 2a11.G1 | NO: 7) | NO: 8) | NO: 9) |
| (L328C)k | SYDMH | VIWYDGS | VIAARTF |
| | | NKYYA | YYYGMDV |
| | | DSLKG | |
| [Ag]AB-5 | SEQUENCE | SEQUENCE | SEQUENCE |
| 75- | (SEQ ID | (SEQ ID | (SEQ ID |
| 2a7.G1 | NO: 10) | NO: 11) | NO: 12) |
| (C99Y; | NYWMS | NIKKDGS | EIQLYLQH |
| L328C)k | | EKFYVD | |
| | | SVKG | |

TABLE 3
VL FR Amino Acid Sequences

| | VL FR1 | VL FR2 | VL FR3 | VL FR4 |
|---|---|---|---|---|
| | (SEQ ID | (SEQ ID | (SEQ ID | (SEQ ID |
| Antibody | NO:) | NO:) | NO:) | NO:) |
| [Ag]AB-4 | SEQUENCE | SEQUENCE | SEQUENCE | SEQUENCE |
| 75- | (SEQ ID | (SEQ ID | (SEQ ID | (SEQ ID |
| 2a1l.G1 | NO: 13) | NO: 14) | NO: 15) | NO: 16) |
| (L32 | DIVMTQS | WYLQKS | GVPDRFS | FGQGTK |
| 8C)k | PLSLPVT | GQSPQ | GSGSGTD | VEIKR |
| | PGEPASI | LLIY | FTLKISR | |
| | SC | | VEAEDVG | |
| | | | VYYC | |
| [Ag]AB-5 | SEQUENCE | SEQUENCE | SEQUENCE | SEQUENCE |
| cHv75- | (SEQ ID | (SEQ ID | (SEQ ID | (SEQ ID |
| 2a7.G1 | NO: 17) | NO: 18) | NO: 19) | NO: 20) |
| (C99 | DIQMTQ | WYQQKPG | GVPSRFS | FGGGTK |
| Y;L328C)k | SPSSVS | KAPKLL | GSGSGTD | VEIKR |
| | ASVGGR | IY | FTLTISN | |
| | VTITC | | LQPEDFA | |
| | | | SYYC | |

TABLE 4
VH FR Amino Acid Sequences

| | VH FR1 | VH FR2 | VH FR3 | VH FR4 |
|---|---|---|---|---|
| | (SEQ ID | (SEQ ID | (SEQ ID | (SEQ ID |
| Antibody | NO:) | NO:) | NO:) | NO:) |
| [Ag]AB-4 | SEQUENCE | SEQUENCE | SEQUENCE | SEQUENCE |
| cHv75- | (SEQ ID | (SEQ ID | (SEQ ID | (SEQ ID |
| 2a11.G1 | NO: 21) | NO: 22) | NO: 23) | NO: 24) |
| (L3 | QVQLVES | WVRQA | RFTISRD | WGQGTT |
| 2SC)k | GGGVVQP | PGKGL | NSKNTLY | VTVSS |
| | GRSLRLS | EWVA | LQMNSLR | |
| | CAASGFT | | AEDTAVY | |
| | FS | | YCAR | |
| [Ag]AB-5 | SEQUENCE | SEQUENCE | SEQUENCE | SEQUENCE |
| 75- | (SEQ ID | (SEQ ID | (SEQ ID | (SEQ ID |
| 2a7.G1 | NO: 25) | NO: 26) | NO: 27) | NO: 28) |
| (C99Y; | EVQLVE | WVRQA | RFTISR | WGQGTL |
| L328C)k | SGGGLV | PGKGL | DNAKNS | VTVSS |
| | QPGGSL | EWVA | LYLQIN | |
| | RLSCAA | | SLRAED | |
| | SGITFS | | TAMYYC | |
| | | | AR | |

TABLE 5
VL Domain Amino Acid Sequences
Variable region (double underline), constant region (dotted underline)

| Antibody | VL (SEQ ID NO:) |
|---|---|
| [Ag]A | SEQUENCE |
| B-4 | (SEQ ID NO: 29) |
| cHv75 | 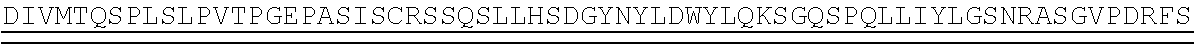 |
| — | 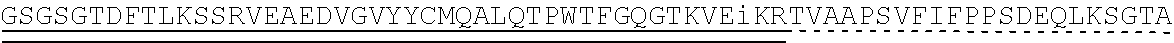 |
| 2a11.G | 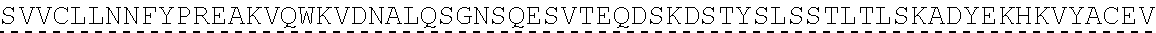 |
| 1(L328 | 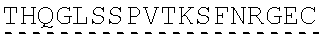 |
| C)k | |
| [Ag]A | SEQUENCE |
| B-5 | (SEQ ID NO: 30) |
| cHv75 | 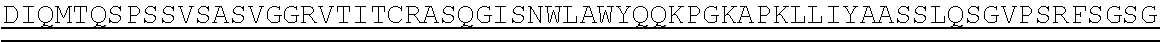 |
| — | 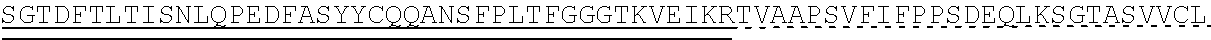 |
| 2a7.G1 | 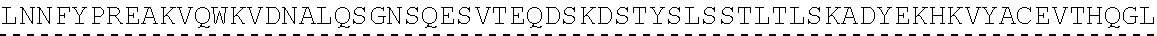 |
| (C99Y; | 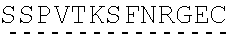 |
| L328 | |
| C)k | |

TABLE 6
VH Domain Amino Add Sequences
Variable region (double underline), constant region (dotted underline)

| Antibody | VH (SEQ ID NO:) |
|---|---|
| [Ag]A | SEQUENCE |
| B-4 | (SEQ ID NO: 31) |
| cHv7 | 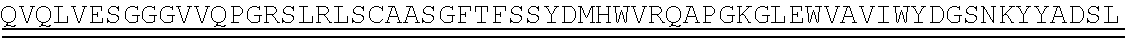 |
| 5- | 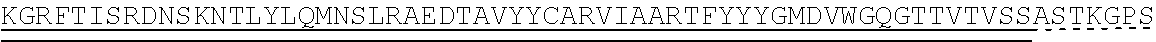 |
| 2all. | 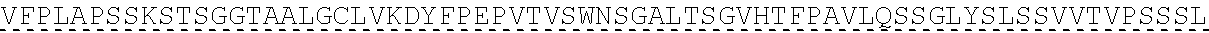 |
| G1(L | 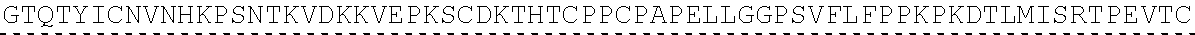 |
| 328C) | 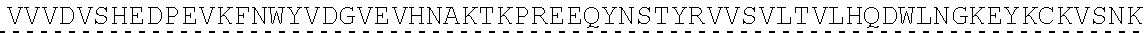 |
| k | 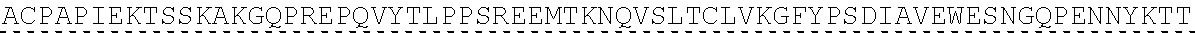 |
| | 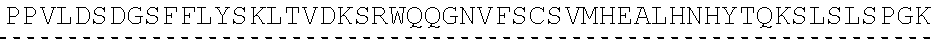 |
| [Ag]A | SEQUENCE |
| B-5 | (SEQ ID NO: 32) |
| cHv7 | 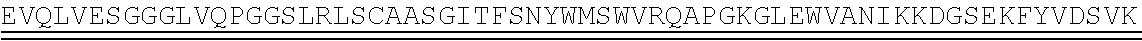 |
| 5- | 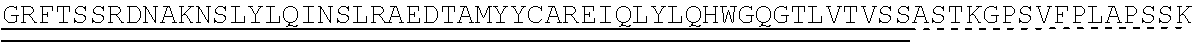 |
| 2a7.G | 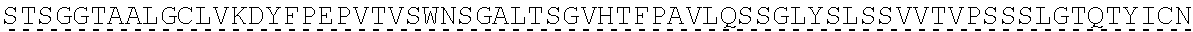 |
| 1(C99 | 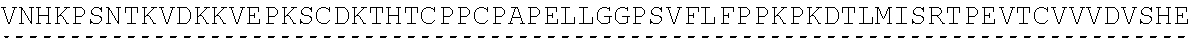 |
| Y;L3 | 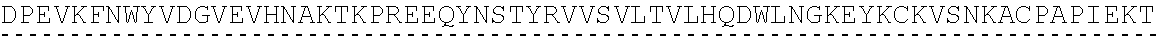 |
| 28C)k | 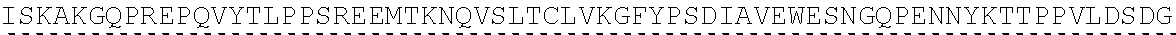 |
| | 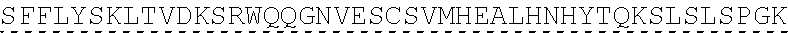 |

TABLE 7
VL Nucleic Acid Sequences

| Antibody | Nucleotide sequences |
|---|---|
| [Ag]A | SEQUENCE |
| B-4 | (SEQ ID NO: 33) |
| cHv7 | GATATTGTGATGACTCAGTC |
| 5- | TCCACTCTCCCTGCCCGTCA |
| 2a11. | CCCCTGGAGAGCCGGCCTCC |
| G1 | ATCTCCTGCAGGTCTAGTCA |
| (L328C) | GAGCCTCCTGCATAGTGATG |
| k | GATACAACTATTTGGATTGG |
| | TACCTGCAGAAGTCAGGGCA |
| | GTCTCCACAGCTCCTGATCT |
| | ATTTGGGTTCTAATCGGGCC |
| | TCCGGGGTCCCTGACAGGTT |
| | CAGTGGCAGTGGATCAGGCA |
| | CAGATTTTACACTGAAAATC |
| | AGCAGAGTGGAGGCTGAGGA |
| | TGTTGGGGTTTATTACTGCA |
| | TGCAAGCTCTACAAACTCCG |
| | TGGACGTTCGGCCAAGGGAC |
| | CAAGGTGGAAATCAAACGGA |
| | CTGTCGCTGCACCATCTGTC |
| | TTCATCTTCCCGCCATCTGA |
| | TGAGCAGTTGAAATCTGGAA |
| | CTGCCTCTGTTGTGTGCCTG |
| | CTGAATAACTTCTATCCCAG |
| | AGAGGCCAAAGTACAGTGGA |
| | AGGTGGATAACGCCCTCCAA |
| | TCGGGTAACTCCCAGGAGAG |
| | TGTCACAGAGCAGGACAGCA |
| | AGGACAGCACCTACAGCCTC |
| | AGCAGCACCCTGACGCTGAG |
| | CAAAGCAGACTACGAGAAAC |
| | ACAAAGTCTACGCCTGCGAA |
| | GTCACCCATCAGGGCCTGAG |
| | CTCGCCCGTCACAAAGAGCT |
| | TCAACAGGGGAGAGTGT |
| [Ag]A | SEQUENCE |
| B-5 | (SEQ ID NO: 34) |
| cHv7 | GACATCCAGATGACCCAGTC |
| 5- | TCCTTCTTCCGTGTCTGCAT |
| 2a7.G | CTGTAGGAGGCAGAGTCACC |
| 1(C99 | ATCACTTGTCGGGCGAGTCA |
| Y;L3 | GGGTATTAGCAACTGGTTAG |
| 28) | CCTGGTATCAGCAGAAACCA |
| | GGGAAAGCCCCTAAACTCCT |
| | GATCTATGCTGCATCCAGTT |
| | TGCAAAGTGGGGTCCCATCA |
| | AGGTTCAGCGGCAGTGGATC |
| | TGGGACAGATTTCACTCTCA |
| | CCATCAGCAACCTGCAGCCT |
| | GAAGATTTTGCAAGTTACTA |
| | TTGTCAACAGGCTAACAGTT |
| | TCCCCCTCACTTTCGGCGGA |
| | GGGACCAAGGTGGAGATCAA |
| | ACGGACTGTCGCTGCACCAT |
| | CTGTCTTCATCTTCCCGCCA |
| | TCTGATGAGCAGTTGAAATC |
| | TGGAACTGCCTCTGTTGTGT |
| | GCCTGCTGAATAACTTCTAT |
| | CCCAGAGAGGCCAAAGTACA |
| | GTGGAAGGTGGATAACGCCC |
| | TCCAATCGGGTAACTCCCAG |
| | GAGAGTGTCACAGAGCAGGA |
| | CAGCAAGGACAGCACCTACA |
| | GCCTCAGCAGCACCCTGACG |
| | CTGAGCAAAGCAGACTACGA |
| | GAAACACAAAGTCTACGCCT |
| | GCGAAGTCACCCATCAGGGC |
| | CTGAGCTCGCCCGTCACAAA |
| | GAGCTTCAACAGGGGAGAGT |
| | GT |

TABLE 8
VH Nucleic Acid Sequences

| Antibody | Nucleotide sequences |
|---|---|
| [Ag]A | SEQUENCE |
| B-4 | (SEQ ID NO: 35) |
| cHv7 | CAGGTGCAGCTGGTGGAGTC |
| 5- | TGGGGGAGGCGTGGTCCAGC |
| 2a11. | CTGGGAGGTCCCTGAGACTC |
| G1(L | TCCTGTGCAGCGTCTGGATT |
| 328C) | CACCTTCAGTAGCTATGACA |
| k | TGCACTGGGTCCGCCAGGCT |
| | CCAGGCAAGGGGCTGGAGTG |
| | GGTGGCAGTTATTTGGTATG |
| | ATGGAAGTAATAAATACTAT |
| | GCAGACTCCTTGAAGGGCCG |
| | ATTCACCATCTCCAGAGACA |
| | ATTCCAAGAACACGCTGTAT |
| | CTGCAAATGAACAGCCTCAG |
| | AGCCGAGGACACGGCTGTGT |
| | ATTACTGTGCGAGGGTTATA |
| | GCAGCTCGTACCTTCTACTA |
| | CTACGGTATGGACGTCTGGG |
| | GCCAAGGGACCACGGTCACC |
| | GTCTCCTCAGCATCCACCAA |
| | GGGCCCATCGGTCTTCCCCC |
| | TGGCACCCTCCTCCAAGAGC |
| | ACCTCTGGGGGCACAGCGGC |
| | CCTGGGCTGCCTGGTCAAGG |
| | ACTACTTCCCCGAACCGGTG |
| | ACGGTGTCGTGGAACTCAGG |
| | CGCCCTGACCAGCGGCGTGC |
| | ACACCTTCCCGGCTGTCCTA |
| | CAGTCCTCAGGACTCTACTC |
| | CCTCAGCAGCGTGGTGACCG |
| | TGCCCTCCAGCAGCTTGGGC |
| | ACCCAGACCTACATCTGCAA |
| | CGTGAATCACAAGCCCAGCA |
| | ACACCAAGGTGGACAAGAAA |
| | GTTGAGCCCAAATCTTGTGA |
| | CAAAACTCACACATGCCCAC |
| | CGTGCCCAGCACCTGAACTC |
| | CTGGGGGGACCGTCAGTCTT |
| | CCTCTTCCCCCCAAAACCCA |
| | AGGACACCCTCATGATCTCC |
| | CGGACCCCTGAGGTCACATG |
| | CGTGGTGGTGGACGTGAGCC |
| | ACGAAGACCCTGAGGTCAAG |
| | TTCAACTGGTACGTGGACGG |
| | CGTGGAGGTGCATAATGCCA |
| | AGACAAAGCCGCGGGAGGAG |
| | CAGTACAACAGCACGTACCG |
| | TGTGGTCAGCGTCCTCACCG |
| | TCCTGCACCAGGACTGGCTG |
| | AATGGCAAGGAGTACAAGTG |
| | CAAGGTCTCCAACAAAGCCT |
| | GCCCAGCCCCCATCGAGAAA |
| | ACCATCTCCAAAGCCAAAGG |
| | GCAGCCCCGAGAACCACAGG |
| | TGTACACCCTGCCCCCATCC |
| | CGGGAGGAGATGACCAAGAA |
| | CCAGGTCAGCCTGACCTGCC |
| | TGGTCAAAGGCTTCTATCCC |
| | AGCGACATCGCCGTGGAGTG |
| | GGAGAGCAATGGGCAGCCGG |
| | AGAACAACTACAAGACCACG |
| | CCTCCCGTGCTGGACTCCGA |
| | CGGCTCCTTCTTCCTCTATA |
| | GCAAGCTCACCGTGGACAAG |
| | AGCAGGTGGCAGCAGGGGAA |
| | CGTCTTCTCATGCTCCGTGA |
| | TGCATGAGGCTCTGCACAAC |
| | CACTACACGCAGAAGAGCCT |
| | CTCCCTGTCTCCGGGTAAA |
| [Ag]A | SEQUENCE |
| B-5 | (SEQ ID IIO: 36) |
| cHv7 | GAGGTGCAGCTGGTGGAGTC |
| 5- | TGGGGGAGGCTTGGTCCAGC |
| 2a7.G | CTGGGGGGTCCCTGAGACTC |
| 1(99 | TCCTGTGCAGCCTCTGGAAT |
| Y;L3 | CACCTTTAGTAATTATTGGA |
| 28C) | TGAGCTGGGTCCGCCAGGCT |
| | CCAGGGAAGGGACTGGAGTG |
| | GGTGGCCAACATAAAGAAAG |
| | ATGGAAGTGAGAAATTCTAT |
| | GTGGACTCTGTGAAGGGCCG |
| | ATTCACCATCTCCAGAGACA |
| | ACGCCAAGAACTCACTGTAT |
| | CTGCAAATCAACAGCCTGAG |
| | AGCCGAGGACACGGCTATGT |
| | ATTACTGTGCGAGAGAAATA |
| | CAGCTATACCTGCAGCACTG |
| | GGGCCAGGGCACCCTGGTCA |
| | CCGTCTCCTCAGCATCCACC |
| | AAGGGCCCATCGGTCTTCCC |
| | CCTGGCACCCTCCTCCAAGA |
| | GCACCTCTGGGGGCACAGCG |
| | GCCCTGGGCTGCCTGGTCAA |
| | GGACTACTTCCCCGAACCGG |
| | TGACGGTGTCGTGGAACTCA |
| | GGCGCCCTGACCAGCGGCGT |
| | GCACACCTTCCCGGCTGTCC |
| | TACAGTCCTCAGGACTCTAC |
| | TCCCTCAGCAGCGTGGTGAC |
| | CGTGCCCTCCAGCAGCTTGG |
| | GCACCCAGACCTACATCTGC |
| | AACGTGAATCACAAGCCCAG |
| | CAACACCAAGGTGGACAAGA |
| | AAGTTGAGCCCAAATCTTGT |
| | GACAAAACTCACACATGCCC |
| | ACCGTGCCCAGCACCTGAAC |
| | TCCTGGGGGGACCGTCAGTC |
| | TTCCTCTTCCCCCCAAAACC |
| | CAAGGACACCCTCATGATCT |
| | CCCGGACCCCTGAGGTCACA |
| | TGCGTGGTGGTGGACGTGAG |
| | CCACGAAGACCCTGAGGTCA |
| | AGTTCAACTGGTACGTGGAC |
| | GGCGTGGAGGTGCATAATGC |
| | CAAGACAAAGCCGCGGGAGG |
| | AGCAGTACAACAGCACGTAC |
| | CGTGTGGTCAGCGTCCTCAC |
| | CGTCCTGCACCAGGACTGGC |
| | TGAATGGCAAGGAGTACAAG |
| | TGCAAGGTCTCCAACAAAGC |
| | CTGCCCAGCCCCCATCGAGA |
| | AAACCATCTCCAAAGCCAAA |
| | GGGCAGCCCCGAGAACCACA |
| | GGTGTACACCCTGCCCCCAT |
| | CCCGGGAGGAGATGACCAAG |
| | AACCAGGTCAGCCTGACCTG |
| | CCTGGTCAAAGGCTTCTATC |
| | CCAGCGACATCGCCGTGGAG |
| | TGGGAGAGCAATGGGCAGCC |
| | GGAGAACAACTACAAGACCA |
| | CGCCTCCCGTGCTGGACTCC |
| | GACGGCTCCTTCTTCCTCTA |
| | TAGCAAGCTCACCGTGGACA |
| | AGAGCAGGTGGCAGCAGGGG |
| | AACGTCTTCTCATGCTCCGT |
| | GATGCATGAGGCTCTGCACA |
| | ACCACTACACGCAGAAGAGC |
| | CTCTCCCTGTCTCCGGGTAA |
| | A |

### EXAMPLES

### EXAMPLE 1. Generation and Characterization of Anti-PSMA Antibodies

To express PSMA antibodies recombinantly in transfected Chinese hamster ovary (CHO) cells, PSMA antibody variable heavy and light chain sequences were cloned into plasmids constructs upstream of the human heavy chain IgG1 and human light chain Igκ constant regions respectively. The complete PSMA antibody human heavy chain and light chain cassettes were cloned downstream of a promoter/enhancer in a cloning vector. A polyadenylation site was included downstream of the antibody coding sequence. The recombinant PSMA antibody expressing constructs were transfected into CHO cells. The protein A purified PSMA antibodies secreted from recombinant CHO cells were evaluated for binding to cell surface PSMA by flow cytometry and by biacore.

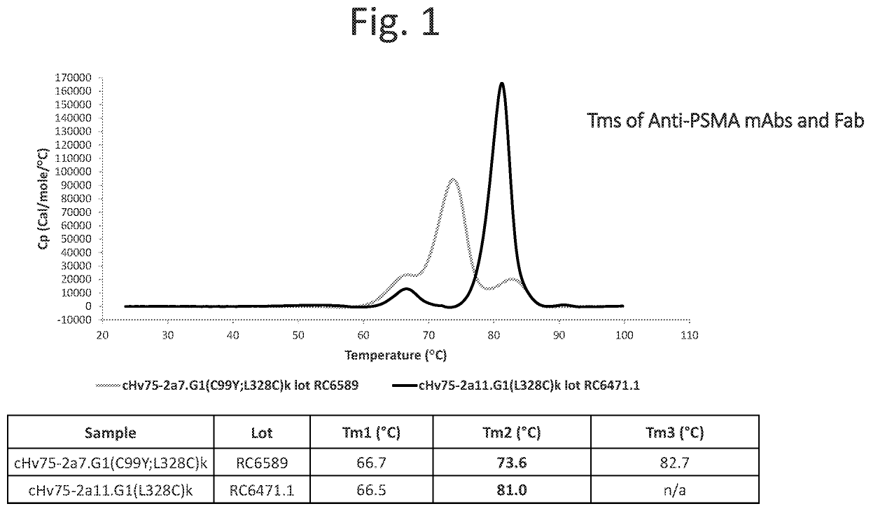

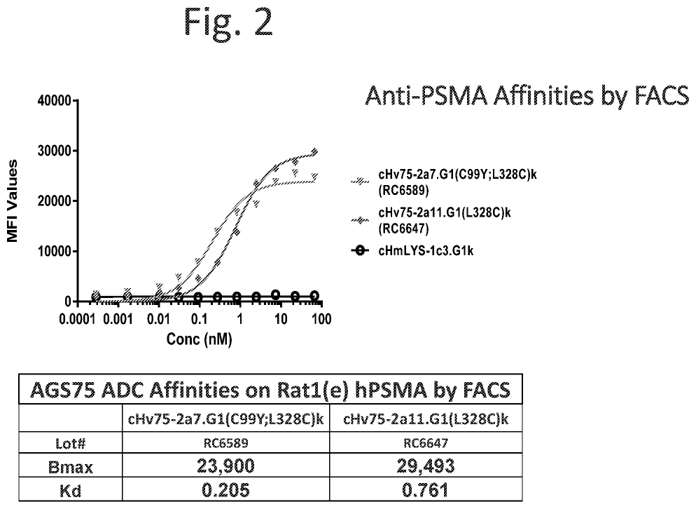

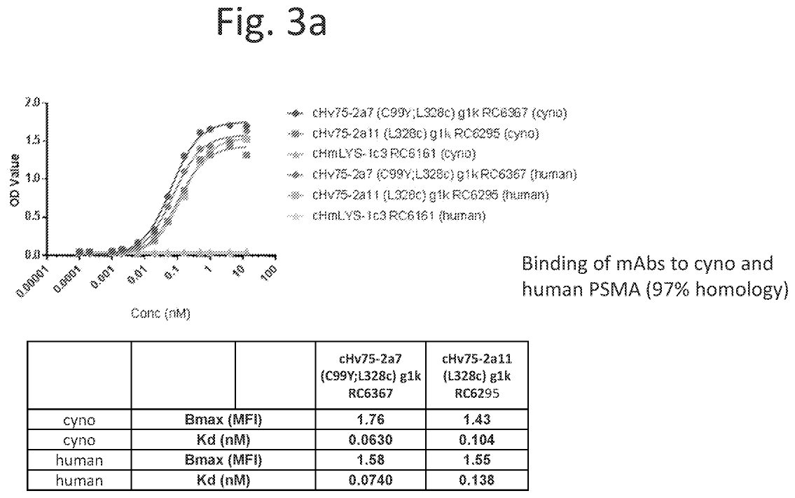

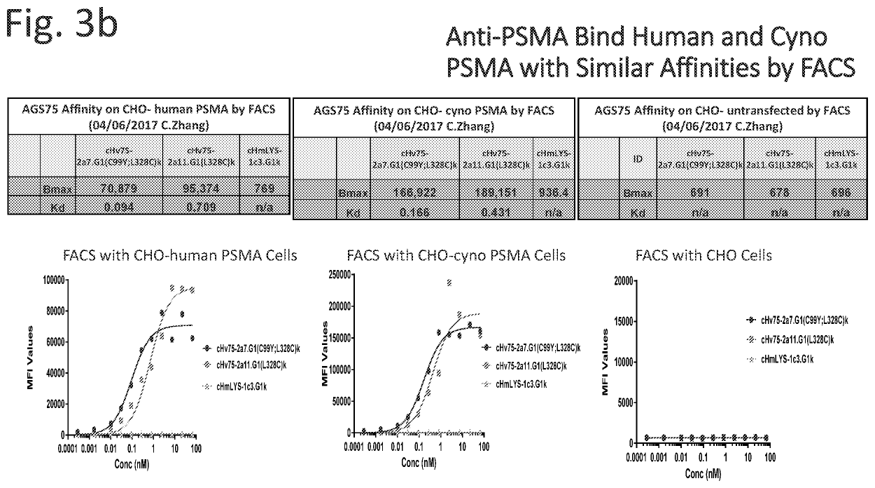

The purified antibodies were subsequently characterized by SDS-PAGE, SEC, CE-SDS, DSC, binding affinity determination, and paralog/homolog binding assessment. FIG. 1 showed DSC assays indicating the Tms of the two anti-PSMA antibodies: Fab Tm of cHv75-2a11.G1(L328C)k was 81.0° C., and Fab Tm of cHv75-2a7.G1(C99Y;L328C)k was 73.6° C. FIG. 2 indicated both antibodies exhibiting good binding affinities on hPSMA. Human and cyno PSMA share 97% homology. The two anti-PSMA antibodies, cHv75-2a1 (L328C)k and cHv75-2a11.G1(1,328C)k, showed similar binding affinities on cyno and human PSMA by ELISA & Biacore (FIG. 3) and FACS (FIG. 3b), but not mouse PSMA.

### EXAMPLE 2. Conjugation and Characterization of Anti-PSMA Antibodies

Flow cells 1 and 2 (FC1 and FC2) were coupled to Protein A/G using the automated immobilization wizard (500 nM Protein A/G in pH 4.5 glycine buffer). Final RUs were 1127 and 1272 respectively.

The strategy was to immobilize the mAbs (25 nM) on FC2 followed by injection of Human PSMA dimer (DT1596) or Cyno PSMA dimer (DT1618) on FC1 and FC2 diluted in HBS-EP buffer at various concentrations (500 nM-31.25 nM).

FC1 was used as reference channel.

Table A showed ADC Yield of the two antibodies.

TABLE A

| | | % | ADC |
|---|---|---|---|
| ADC Name | DAR | Aggregate | Yield (%) |
| cHv75-2a7.G1(C99Y; | 1.86 | 1.13 | 93.3 |
| L328C)k-AGL-01332-93 | | | |
| cHv75-2a11.G1(L328C)k- | 1.93 | 0.93 | 99.6 |
| AGL-01332-93 | | | |

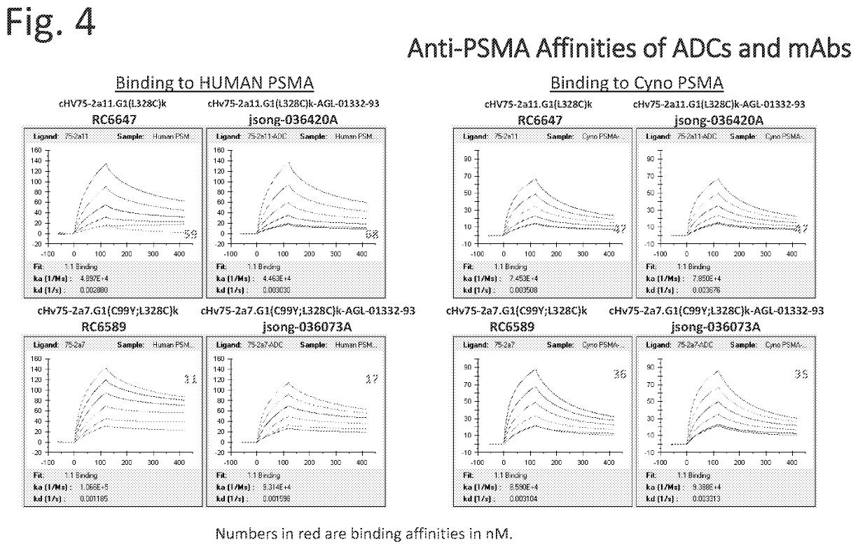

FIG. 4 compared binding affinities of anti-PSMA antibodies and conjugated antibodies to PSMA, indicating that conjugations do not affect PSMA binding.

AGS75 ADC was dosed at 5 mg/kg as a single intravenous bolus injection on day 0 into female CD17/SCID non-tumor bearing mice. Blood samples were collected at different time points starting 2 minutes post injection up to 21 days after dosing. Serum was collected immediately after complete clotting and stored frozen until the analysis.

The PK ECL followed a standard sandwich ELISA technique, with PSMA protein being used as the capture protein. In brief, assay plates (Standard MSD plates) were coated with 50 μl of PSMA at a concentration of 1 μg/ml and incubated overnight at 4° C. On day 2, the coating solution was washed with PBS/0.05% Tween20 wash buffer using the plate washer. 150 μL of blocking buffer was added and incubated at room temp for 1 hour followed by 3 washes with 300 μl/well of PBS/0.05% Tween20 using the plate washer. Serially diluted standard and serum study samples are pipetted into the wells. The 12-point standard curve in 1% Mouse; serum samples tested at 1:16200 dilution, ran in duplicates (50 μl/well). The controls were also added in duplicate. The plates were covered and incubated for 1 hour at room temp, then washed 3 times to remove the excess unbound substances and 50 μl/well of MSD Anti-Human IgG3 SulfoTag detection antibody for total protein and 50 μl/well of SG15.22 for ADC added in assay buffer. The plates were covered, incubated for 1 hour at room temp, washed 3 times. For total assay, 150 μl/well of MSD Read buffer (diluted to 2×with D.I. water) is added to the wells. The plates were then read on the MSD Meso Sector S600. For ADC assay, 50 μl/well of diluted MSD Streptavidine sulfa-tag was added. The plates were covered and incubated for 1 hour at room temp, then washed 3 times to remove the excess unbound detection antibody and 150 μl/well of MSD Read buffer (diluted to 2×with D.I. water) is added to the wells. The plates were then read on the MSD Meso Sector S600 and analyzed via MSD Discovery Workbench software.

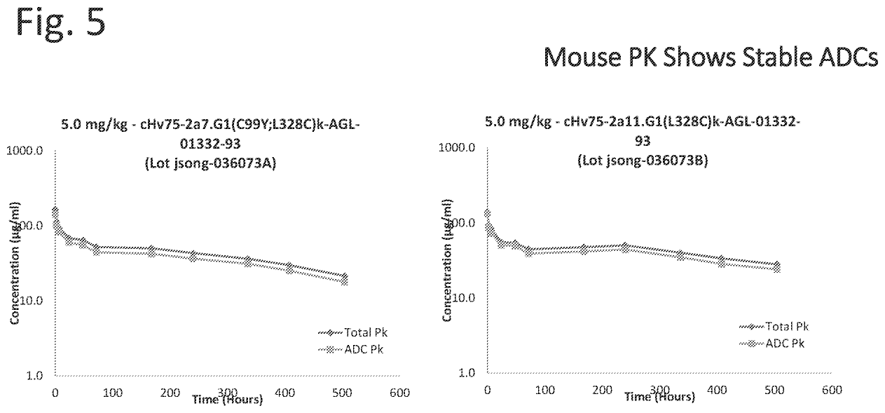

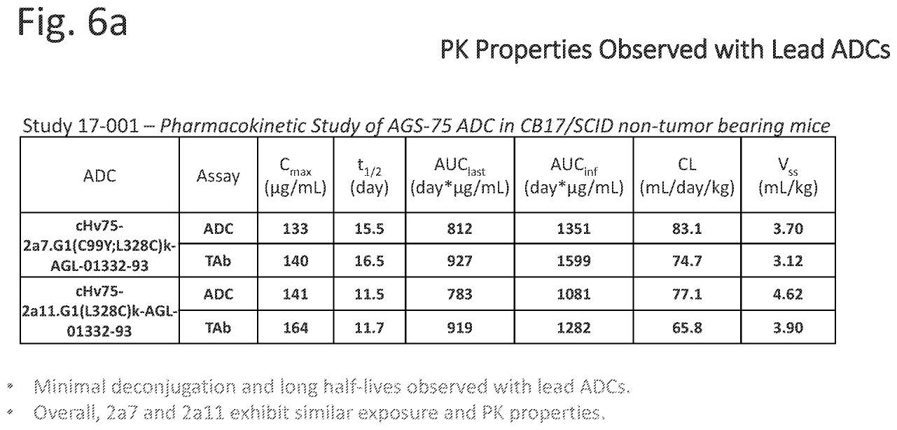

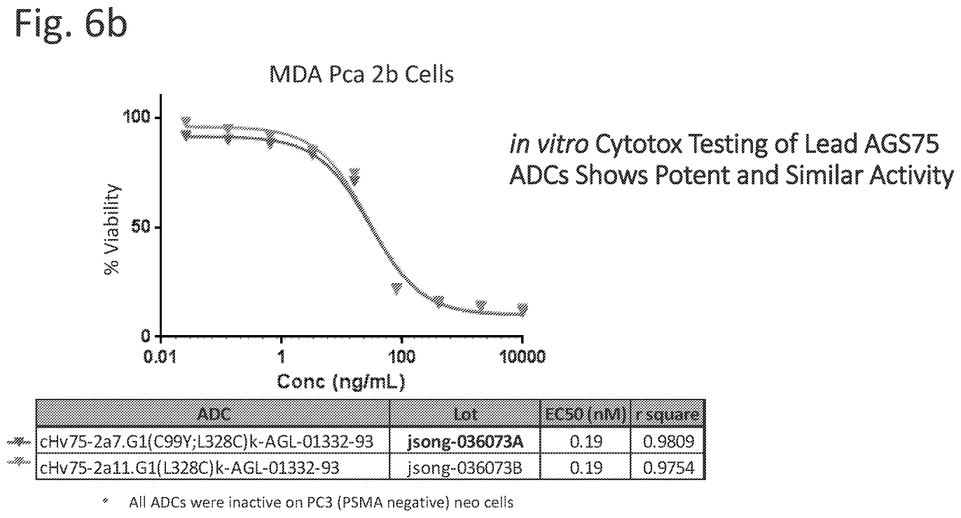

FIG. 5 showed stability of ADCs. Table B and FIG. 6a showed Pharmacokinetic Study of AGS-75 ADC, in CB17/SCID non-tumor bearing mice. Minimal deconjugation and long half-lives were observed with both ADCs, and 2a7 and 2a11 exhibit similar exposure and PK properties.

TABLE B

| | | | | AUCₗₐₛₜ | AUC_{inf} | CL | |
|---|---|---|---|---|---|---|---|
| | | Cₘₐₓ | | (day * | (day * | (mL/ | Vₛₛ |
| | | (μg/ | t_{1/2} | μg/ | μg/ | day/ | (mL/ |
| ADC | Assay | mL) | (day) | mL) | ml) | kg) | kg) |
| cHv75- | ADC | 133 | 15.5 | 812 | 1351 | 83.1 | 3.70 |
| 2a7.G1(C99Y; | TAb | 140 | 16.5 | 927 | 1599 | 74.7 | 3.12 |
| L328C) | | | | | | | |
| k-AGL-01332-93 | | | | | | | |
| cHv75- | ADC | 141 | 11.5 | 783 | 1081 | 77.1 | 4.62 |
| 2a11.G1(L328C) | TAb | 164 | 11.7 | 919 | 1282 | 65.8 | 3.90 |
| k-AGL-01332-93 | | | | | | | |

### EXAMPLE 3. In vitro Cytotoxicity of Conjugated Anti-PSMA Antibodies

To assess in vitro cytotoxicity with PSMA directed 01332-93 ADC, MDA Pca 2b cells were plated at 5000 cells/well in F-12 media (Gibco) with supplements in 96 well plates. After overnight culture at 37 degrees ADC were titrated into the cultures starting at 5 ug/mL. Cells were cultured with ADC for 6 days and cell viability was assessed by Cell Titre Glo (Promega) assay after 10′ incubation. Luminescence was determined on a Synergy plate reader (BioTek). % Survival vs. ADC concentration curves and EC50s were calculated with Graph Pad Prism software.

FIG. 6b indicated both ADCs exhibited similar and potent cytotoxicity.

### EXAMPLE 4. In vivo Efficacy of Conjugated Anti-PSMA Antibodies in a LAPC9AI Tumor Mouse Model

Two to five pieces of LAPC9AI or LNCaP tumors were implanted subcutaneously per male CB17/SCID or NSG mice 4-6 weeks of age. When the average tumor volumes reached approximately 200 mm³, mice were size matched and randomized into treatment and control groups before giving a single dose of AGS75 ADC intravenously at 2.5 mg/kg, 4 mg/kg and 6 mg/kg for each treatment group. Tumor size was determined by external caliper measurement twice a week.

A statistical analysis of the tumor volume data was performed using the Kruskal-Wallis test and the implementation of the Kruskal-Wallis test was carried out using the parametric ANOVA F-test on the ranks of the data. The percent tumor growth inhibition in each treated group versus a control group was calculated as [(Control−Control baseline)−(Treated−Treated baseline)]/(Control−Control baseline)×100%. The percent of tumor regression was defined as (Treated baseline-Treated)/Treated baseline×100%.

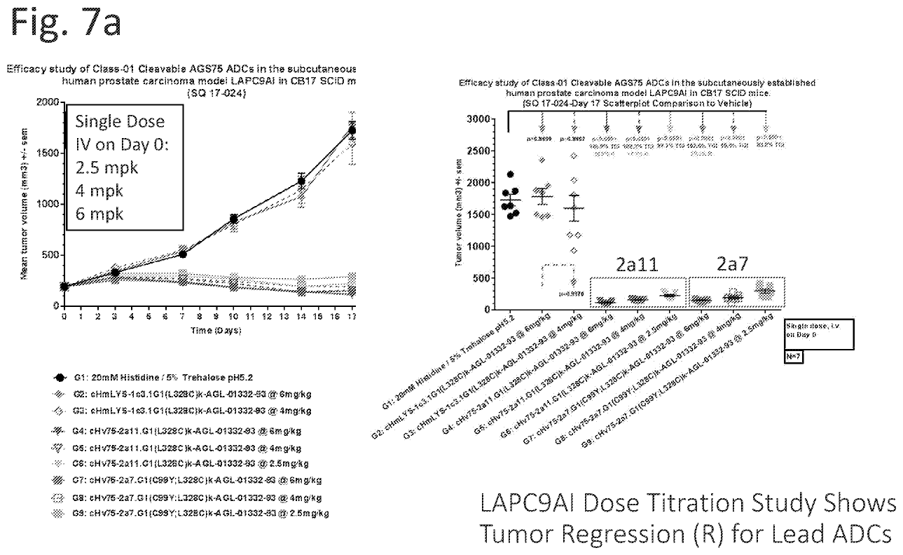

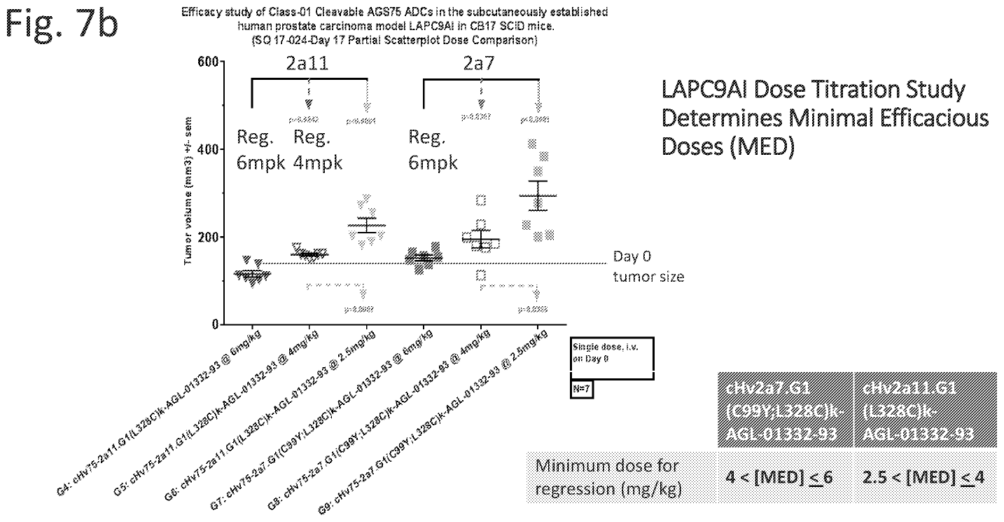

FIG. 7a showed tumor regression after administrating ADCs into the subcutaneout human prostate carcinoma model LAPC9AI in CB17 SCID mice. FIG. 7h showed the minimum dose of ADCs for tumor regression (mg/kg).

### EXAMPLE 5. In Vivo Efficacy of Conjugated Anti-PSMA Antibodies in a LNCaP Tumor Mouse Model

Two to five pieces of LAPC9AI or LNCaP tumors were implanted subcutaneously per male CB17/SCID or NSG mice 4-6 weeks of age. When the average tumor volumes reached approximately 200 mm³, mice were size matched and randomized into treatment and control groups before giving a single dose of AGS75 ADC intravenously at 2.5 mg/kg, 4 mg/kg and 6 mg/kg for each treatment group. Tumor size was determined by external caliper measurement twice a week.

A statistical analysis of the tumor volume data was performed using the Kruskal-Wallis test and the implementation of the Kruskal-Wallis test was carried out using the parametric ANOVA F-test on the ranks of the data. The percent tumor growth inhibition in each treated group versus a control group was calculated as [(Control−Control baseline)−(Treated−Treated baseline)]/(Control−Control baseline)×100%. The percent of tumor regression was defined as (Treated baseline−Treated)/Treated baseline×100%.

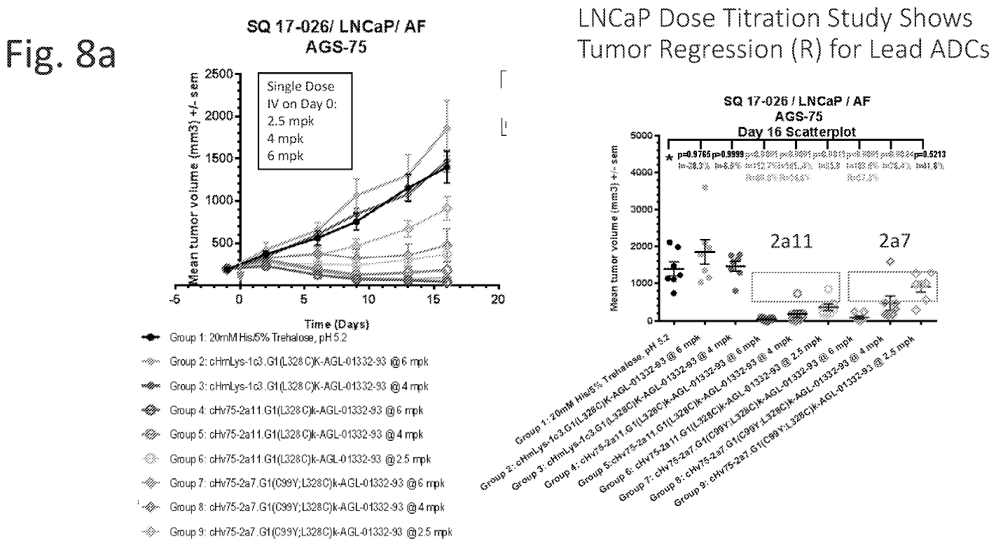

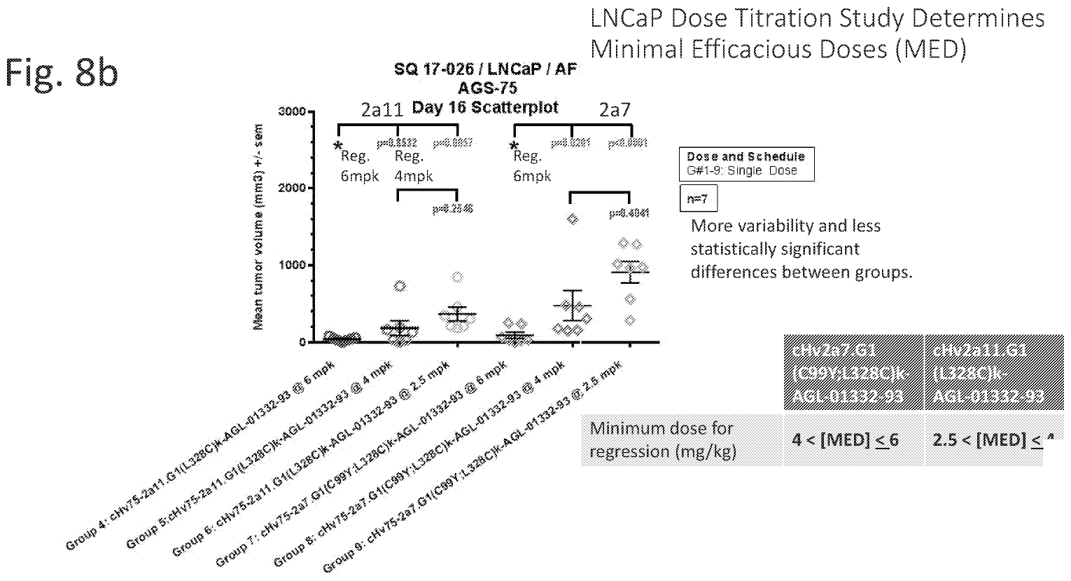

FIG. 8a showed tumor regression after administrating ADCs into the subcutaneout human prostate carcinoma model LNCaP in CB17 SCID mice. FIG. 8b showed the minimum dose of ADCs for tumor regression (mg/kg).

### EXAMPLE 6. In Vivo Toxicity Study of Conjugated Anti-PSMA Antibodies in a Cynomolgos Model

To determine on-target and off-target toxicity profile of cHv75-2a11.G1(L328C)k-AGL-01332-93 and potential recovery.

Groups: control, cHv75-2a11.G1(L1328C)k-AGL-01332-93 at 9 & 12 mg/kg

Animals on study: n=4/dose level (2/gender), 1/gender/dose level at each the dosing and recovery phase necropsies

endpoints: body weight, food consumption, clinical observations, clinical pathology, urinalysis, BA/TK samples

Postmortem endpoints: gross necropsy, anatomic pathology (terminal and recovery)

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following.

What is claimed:

1. An isolated antibody or an antigen binding fragment thereof (AB) that specifically binds to mammalian PSMA, wherein the AB specifically binds human PSMA and cynomolgus monkey PSMA, wherein the antibody or antigen-binding fragment thereof comprises a VH CDR1 amino acid sequence SYDMH (SEQ ID NO: 7); a VH CDR2 amino acid sequence VIWYDGSNKYYADSLKG (SEQ ID NO: 8); a VH CDR3 amino acid sequence VIAARTFYYYGMDV (SEQ ID NO: 9); a VL CDR1 amino acid sequence RSSQSLLHSDGYNYLD (SEQ ID NO: 1); a VL CDR2 amino acid sequence LGSNRAS (SEQ ID NO: 2); and a VL CDR3 amino acid sequence MQALQTPWT (SEQ ID NO: 3).

2. The isolated antibody of claim 1, wherein the AB comprises a heavy chain variable region comprising the amino acid sequence of SEQ ID NO: 31, and a light chain variable region comprising an amino acid sequence selected from the group consisting of SEQ NOs: 29.

3. The isolated antibody of claim 1 or claim 2, wherein the antigen binding fragment thereof is selected from the group consisting of a Fab fragment, a F(ab′)2 fragment, a scFv, and a scAb.

4. The isolatedantibody of any one of claims 1 to 3, wherein the AB specifically binds human PSMA.

5. A conjugated antibody comprising the isolated antibody of any one of claims 1 to 4 conjugated to an agent.

6. The conjugated antibody of claim 5, wherein the agent is a toxin or fragment thereof.

7. The conjugated antibody of claim 6, wherein the agent is a microtubule inhibitor.

8. The conjugated antibody of claim 7, wherein the agent is selected from the group consisting of a dolastatin or a derivative thereof, an auristatin or a derivative thereof, a maytansinoid or a derivative thereof, a duocarmycin or a derivative thereof, a calicheamicin or a derivative thereof, and a pyrrolobenzodiazepine or a derivative thereof.

9. The conjugated antibody of claim 8, wherein the agent comprises a molecule having a structure of formula (1): 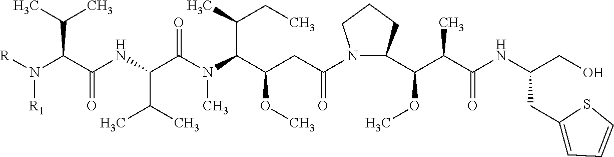
wherein R1 is a hydrogen or a C16 alkyl group;
wherein R is selected from the group consisting of: a hydrogen, a C₁₋₆ a linker, or a group X1-Y1-* wherein * is the point of attachment to the nitrogen; and
wherein Y1 is an oxycarbonyl group and X1 is a C₁₋₆ alkyl group, a 9-fluorenylmethyl group, a benzyl group, or a tert-butyl group.

10. The conjugated antibody of any one of claims 5 to 9, wherein the agent is conjugated to the AB via a linker.

11. The conjugated antibody of claim 10, wherein the linker with a structure of formula (II): 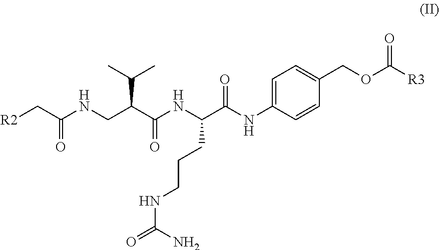
wherein R3 is an agent attached to formula (II) where the point of attachment is a nitrogen, sulfur, oxygen, or carbon atom; and
wherein R2 is a moiety attached to formula (II) wherein the point of attachment is selected from the group consisting of: a chlorine group, an iodine group, a bromine group, and a thiol group.

12. The conjugated antibody of claim 10, wherein the linker is a cleavable linker.

13. The conjugated antibody of claim 10, wherein the linker is a non-cleavable linker.

14. The conjugated antibody of claim 5, wherein the agent is a detectable moiety.

15. The conjugated antibody of claim 14, wherein the detectable moiety is a diagnostic agent.

16. The conjugated antibody of any one of claims 5 to 15, wherein the mammalian PSMA is a human PSMA and cynomolgus monkey PSMA.

17. The conjugated antibody of claim 5, wherein the agent is conjugated to the AB via a linker, and wherein the linker and the toxin have the structure of formula (III): 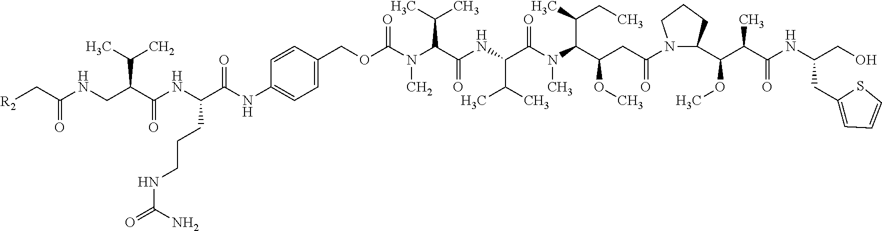
wherein R2 is a point of attachment to the AB.

18. The conjugated antibody of any one of claims 5 to 17, wherein the agent is conjugated to a thiol group on the AB.

19. The conjugated antibody of claim 18, wherein the thiol group is a cysteine side chain thiol group.

20. The conjugated antibody of claim 19, wherein the cysteine residue of the conjugated thiol group is at Kabat position 328 of the AB.

71. A conjugated antibody comprising:
an antibody or antigen binding fragment thereof (AB) that specifically binds to mammalian PSMA, wherein the AB comprises the VH CDR1 amino acid sequence SYDMH (SEQ ID NO: 7); the VH CDR2 amino acid sequence VIWYDGSNKYYADSLKG (SEQ ID NO: 8); the VH CDR3 amino acid sequence VIAARTFYYYGMDV (SEQ ID NO: 9); the VL CDR1 amino acid sequence RSSQSLLHSDGYNYLD (SEQ ID NO: 1); the VL CDR2 amino acid sequence LGSNRAS (SEQ ID NO: 2); and the VL CDR3 amino acid sequence MQALQTPWT (SEQ ID NO: 3); and
an agent conjugated to the AB, wherein the agent comprises a molecule having a structure of formula (I): 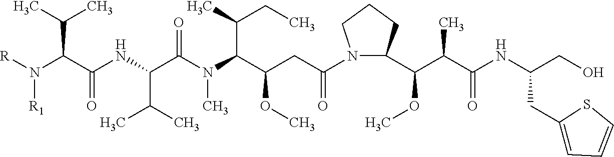
wherein R1 is a hydrogen or a C₁₋₆ alkyl group;
wherein R is selected from the group consisting of: a hydrogen, a C₁₋₆ alkyl, a linker, or a group X1-Y1-* wherein * is the point of attachment to the nitrogen; and
wherein Y1 is an oxycarbonyl group and X1 is a C₁₋₆ alkyl group, a 9-fluorenylmethyl group, a benzyl group, or a tert-butyl group.

22. The conjugated antibody of claim 21, wherein the AB comprises a heavy chain variable region comprising an amino acid sequence of SEQ ID NO: 31, and a light chain variable region comprising an amino acid sequence of SEQ ID NO: 29.

23. The conjugated antibody of claim 21 or claim 22, wherein the agent is conjugated to the AB via a linker.

24. The conjugated antibody of claim 23, wherein the linker with a structure of formula (II): 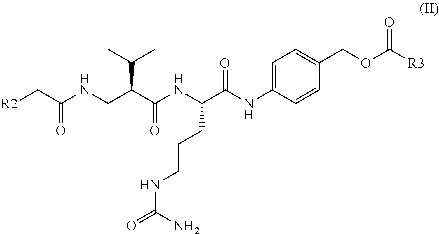
wherein R3 is point of attachment to the molecule of formula (I); and
wherein R2 is the point of attachment to the AB.

25. The conjugated antibody of claim 23, wherein the linker and the agent has a structure of formula (III): 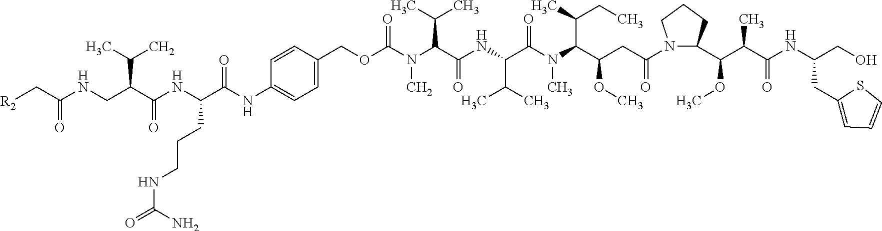
wherein R2 is the point of attachment is to the AB.

26. A pharmaceutical composition comprising the isolated antibody of any one of claims 1 to 4, or the conjugated antibody of any one of claims 5 to 25, and a carrier.

27. The pharmaceutical composition of claim 26 comprising an additional agent.

28. The pharmaceutical composition of claim 27, wherein the additional agent is a therapeutic agent.

29. An isolated nucleic acid molecule encoding the isolated antibody of any one of claims 1 to 4.

30. A vector comprising the isolated nucleic acid molecule of claim 29.

31. A method of producing an antibody by culturing a cell under conditions that lead to expression of the antibody, wherein the cell comprises the nucleic acid molecule of claim 29 or the vector of claim 30.

32. A method of manufacturing an antibody that binds PSMA, the method comprising: (a) culturing a cell comprising a nucleic acid construct that encodes the antibody of any one of claims 1 to 4 under conditions that lead to expression of the antibody; and (b) recovering the antibody.

33. A method of treating, alleviating a symptom of, or delaying the progression of a disorder or disease in which diseased cells express PSMA, comprising administering a therapeutically effective amount of the antibody of any one of claims 1 to 4, the conjugated antibody of any one of claims 5 to 25, or the pharmaceutical composition of claim 26 to a subject in need thereof.

34. A method of treating, alleviating a symptom of, or delaying the progression of a disorder or disease associated with cells expressing PSMA comprising administering a therapeutically effective amount of the antibody of any one of claims 1 to 4, the conjugated antibody of any one of claims 5 to 25, or the pharmaceutical composition of claim 26 to a subject in need thereof.

35. The method of claim 33 or claim 34, wherein the disorder or disease associated with cells expressing PSMA is cancer.

36. The method of claim 35, wherein the cancer is a prostate cancer or a metastatic castration-resistant prostate carcinoma.

37. A method of inhibiting or reducing the growth, proliferation, or metastasis of cells expressing mammalian PSMA comprising administering a therapeutically effective amount of the antibody of any one of claims 1 to 4, the conjugated antibody of any one of claims 5 to 25, or the pharmaceutical composition of claim 26 to a subject in need thereof.

38. A method of inhibiting, blocking, or preventing the binding of a natural ligand or receptor to mammalian PSMA, comprising administering a therapeutically effective amount of the antibody of any one of claims 1 to 4, the conjugated antibody of any one of claims 5 to 25, or the pharmaceutical composition of claim 26 to a subject in need thereof.

39. The method of any one of claims 33 to 38, wherein the method comprises administering an additional agent.

40. The method of claim 39, wherein the additional agent is a therapeutic agent.


SEQUENCE LISTING (72 sequences)

SEQ ID NO: 1 - protein, 16 aa, Artificial Sequence
  Synthetic
RSSQSLLHSDGYNYLD

SEQ ID NO: 2 - protein, 7 aa, Artificial Sequence
  Synthetic
LGSNRAS

SEQ ID NO: 3 - protein, 9 aa, Artificial Sequence
  Synthetic
MQALQTPWT

SEQ ID NO: 4 - protein, 11 aa, Artificial Sequence
  Synthetic
RASQGISNWLA

SEQ ID NO: 5 - protein, 7 aa, Artificial Sequence
  Synthetic
AASSLQS

SEQ ID NO: 6 - protein, 9 aa, Artificial Sequence
  Synthetic
QQANSFPLT

SEQ ID NO: 7 - protein, 5 aa, Artificial Sequence
  Synthetic
SYDMH

SEQ ID NO: 8 - protein, 17 aa, Artificial Sequence
  Synthetic
VIWYDGSNKYYADSLKG

SEQ ID NO: 9 - protein, 14 aa, Artificial Sequence
  Synthetic
VIAARTFYYYGMDV

SEQ ID NO: 10 - protein, 5 aa, Artificial Sequence
  Synthetic
NYWMS

SEQ ID NO: 11 - protein, 17 aa, Artificial Sequence
  Synthetic
NIKKDGSEKFYVDSVKG

SEQ ID NO: 12 - protein, 8 aa, Artificial Sequence
  Synthetic
EIQLYLQH

SEQ ID NO: 13 - protein, 23 aa, Artificial Sequence
  Synthetic
DIVMTQSPLSLPVTPGEPASISC

SEQ ID NO: 14 - protein, 15 aa, Artificial Sequence
  Synthetic
WYLQKSGQSPQLLIY

SEQ ID NO: 15 - protein, 32 aa, Artificial Sequence
  Synthetic
GVPDRFSGSGSGTDFTLKISRVEAEDVGVYYC

SEQ ID NO: 16 - protein, 11 aa, Artificial Sequence
  Synthetic
FGQGTKVEIKR

SEQ ID NO: 17 - protein, 23 aa, Artificial Sequence
  Synthetic
DIQMTQSPSSVSASVGGRVTITC

SEQ ID NO: 18 - protein, 15 aa, Artificial Sequence
  Synthetic
WYQQKPGKAPKLLIY

SEQ ID NO: 19 - protein, 32 aa, Artificial Sequence
  Synthetic
GVPSRFSGSGSGTDFTLTISNLQPEDFASYYC

SEQ ID NO: 20 - protein, 11 aa, Artificial Sequence
  Synthetic
FGGGTKVEIKR

SEQ ID NO: 21 - protein, 30 aa, Artificial Sequence
  Synthetic
QVQLVESGGGVVQPGRSLRLSCAASGFTFS

SEQ ID NO: 22 - protein, 14 aa, Artificial Sequence
  Synthetic
WVRQAPGKGLEWVA

SEQ ID NO: 23 - protein, 32 aa, Artificial Sequence
  Synthetic
RFTISRDNSKNTLYLQMNSLRAEDTAVYYCAR

SEQ ID NO: 24 - protein, 11 aa, Artificial Sequence
  Synthetic
WGQGTTVTVSS

SEQ ID NO: 25 - protein, 30 aa, Artificial Sequence
  Synthetic
EVQLVESGGGLVQPGGSLRLSCAASGITFS

SEQ ID NO: 26 - protein, 14 aa, Artificial Sequence
  Synthetic
WVRQAPGKGLEWVA

SEQ ID NO: 27 - protein, 32 aa, Artificial Sequence
  Synthetic
RFTISRDNAKNSLYLQINSLRAEDTAMYYCAR

SEQ ID NO: 28 - protein, 11 aa, Artificial Sequence
  Synthetic
WGQGTLVTVSS

SEQ ID NO: 29 - protein, 219 aa, Artificial Sequence
  Synthetic
DIVMTQSPLSLPVTPGEPASISCRSSQSLLHSDGYNYLDWYLQKSGQSPQLLIYLGSNRASGVPDRFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, Artificial Sequence
  Synthetic
DIQMTQSPSSVSASVGGRVTITCRASQGISNWLAWYQQKPGKAPKLLIYAASSLQSGVPSRFSGSGSGTDFTLTISNLQPEDFASYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 453 aa, Artificial Sequence
  Synthetic
QVQLVESGGGVVQPGRSLRLSCAASGFTFSSYDMHWVRQAPGKGLEWVAVIWYDGSNKYYADSLKGRFTISRDNSKNTLYLQMNSLRAEDTAVYYCARVIAARTFYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 447 aa, Artificial Sequence
  Synthetic
EVQLVESGGGLVQPGGSLRLSCAASGITFSNYWMSWVRQAPGKGLEWVANIKKDGSEKFYVDSVKGRFTISRDNAKNSLYLQINSLRAEDTAMYYCAREIQLYLQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 657 bp, Artificial Sequence
  Synthetic
gatattgtgatgactcagtctccactctccctgcccgtcacccctggagagccggcctccatctcctgcaggtctagtcagagcctcctgcatagtgatggatacaactatttggattggtacctgcagaagtcagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtggacgttcggccaagggaccaaggtggaaatcaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 34 - DNA, 642 bp, Artificial Sequence
  Synthetic
gacatccagatgacccagtctccttcttccgtgtctgcatctgtaggaggcagagtcaccatcacttgtcgggcgagtcagggtattagcaactggttagcctggtatcagcagaaaccagggaaagcccctaaactcctgatctatgctgcatccagtttgcaaagtggggtcccatcaaggttcagcggcagtggatctgggacagatttcactctcaccatcagcaacctgcagcctgaagattttgcaagttactattgtcaacaggctaacagtttccccctcactttcggcggagggaccaaggtggagatcaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 35 - DNA, 1359 bp, Artificial Sequence
  Synthetic
caggtgcagctggtggagtctgggggaggcgtggtccagcctgggaggtccctgagactctcctgtgcagcgtctggattcaccttcagtagctatgacatgcactgggtccgccaggctccaggcaaggggctggagtgggtggcagttatttggtatgatggaagtaataaatactatgcagactccttgaagggccgattcaccatctccagagacaattccaagaacacgctgtatctgcaaatgaacagcctcagagccgaggacacggctgtgtattactgtgcgagggttatagcagctcgtaccttctactactacggtatggacgtctggggccaagggaccacggtcaccgtctcctcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - DNA, 1341 bp, Artificial Sequence
  Synthetic
gaggtgcagctggtggagtctgggggaggcttggtccagcctggggggtccctgagactctcctgtgcagcctctggaatcacctttagtaattattggatgagctgggtccgccaggctccagggaagggactggagtgggtggccaacataaagaaagatggaagtgagaaattctatgtggactctgtgaagggccgattcaccatctccagagacaacgccaagaactcactgtatctgcaaatcaacagcctgagagccgaggacacggctatgtattactgtgcgagagaaatacagctatacctgcagcactggggccagggcaccctggtcaccgtctcctcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 37 - protein, 11 aa, Artificial Sequence
  Synthetic
RASQSISRFLN

SEQ ID NO: 38 - protein, 7 aa, Artificial Sequence
  Synthetic
VTSSLQS

SEQ ID NO: 39 - protein, 9 aa, Artificial Sequence
  Synthetic
QQSYNTPIT

SEQ ID NO: 40 - protein, 11 aa, Artificial Sequence
  Synthetic
RASQSIGTFLN

SEQ ID NO: 41 - protein, 7 aa, Artificial Sequence
  Synthetic
VASSLQS

SEQ ID NO: 42 - protein, 9 aa, Artificial Sequence
  Synthetic
QQSYSVPIT

SEQ ID NO: 43 - protein, 5 aa, Artificial Sequence
  Synthetic
SYVMH

SEQ ID NO: 44 - protein, 17 aa, Artificial Sequence
  Synthetic
GVSSSGDSTFYVDSVKG

SEQ ID NO: 45 - protein, 12 aa, Artificial Sequence
  Synthetic
GGITGAPLVFDI

SEQ ID NO: 46 - protein, 5 aa, Artificial Sequence
  Synthetic
SHIMY

SEQ ID NO: 47 - protein, 17 aa, Artificial Sequence
  Synthetic
GISSNGLSSYYVDSVKG

SEQ ID NO: 48 - protein, 12 aa, Artificial Sequence
  Synthetic
GGRDRVPAVFDY

SEQ ID NO: 49 - protein, 23 aa, Artificial Sequence
  Synthetic
DIQMTQSPSSLSASVGDRVTITC

SEQ ID NO: 50 - protein, 15 aa, Artificial Sequence
  Synthetic
WYQQKPGKAPKVLIY

SEQ ID NO: 51 - protein, 32 aa, Artificial Sequence
  Synthetic
GVPSRFSGSGSGTDFTLTISSLQPEDFATYYC

SEQ ID NO: 52 - protein, 11 aa, Artificial Sequence
  Synthetic
FGQGTRLEIKR

SEQ ID NO: 53 - protein, 23 aa, Artificial Sequence
  Synthetic
DIQMTQSPSSLSASIGDRVTITC

SEQ ID NO: 54 - protein, 15 aa, Artificial Sequence
  Synthetic
WYQQKPGKAPKVLIY

SEQ ID NO: 55 - protein, 32 aa, Artificial Sequence
  Synthetic
GVPSRFIGSGSGTDFTLTISSLQPEDFATYYC

SEQ ID NO: 56 - protein, 11 aa, Artificial Sequence
  Synthetic
FGQGTRLEIKR

SEQ ID NO: 57 - protein, 30 aa, Artificial Sequence
  Synthetic
EVQLVESGGGLVQPGGSLRLSCAASGFTFS

SEQ ID NO: 58 - protein, 14 aa, Artificial Sequence
  Synthetic
WVRQAPGKGLEYVS

SEQ ID NO: 59 - protein, 32 aa, Artificial Sequence
  Synthetic
RFTISRDNSKNTLYLQMGSLRAEDMAVYYCAR

SEQ ID NO: 60 - protein, 11 aa, Artificial Sequence
  Synthetic
WGQGTMVTVSS

SEQ ID NO: 61 - protein, 30 aa, Artificial Sequence
  Synthetic
EVQLVESGGGWVQPGGSLRLSCAASGFTFS

SEQ ID NO: 62 - protein, 14 aa, Artificial Sequence
  Synthetic
WVRQAPGKGLEYVS

SEQ ID NO: 63 - protein, 32 aa, Artificial Sequence
  Synthetic
RFTISRDNSKNLLYVHMGSLKPEDMAMYYCAR

SEQ ID NO: 64 - protein, 11 aa, Artificial Sequence
  Synthetic
WGQGTLVTVSS

SEQ ID NO: 65 - protein, 214 aa, Artificial Sequence
  Synthetic
DIQMTQSPSSLSASVGDRVTITCRASQSISRFLNWYQQKPGKAPKVLIYVTSSLQSGVPSRFSGSGSGTDFTLTISSLQPEDFATYYCQQSYNTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, Artificial Sequence
  Synthetic
DIQMTQSPSSLSASIGDRVTITCRASQSIGTFLNWYQQKPGKAPKVLIYVASSLQSGVPSRFIGSGSGTDFTLTISSLQPEDFATYYCQQSYSVPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 451 aa, Artificial Sequence
  Synthetic
EVQLVESGGGLVQPGGSLRLSCAASGFTFSSYVMHWVRQAPGKGLEYVSGVSSSGDSTFYVDSVKGRFTISRDNSKNTLYLQMGSLRAEDMAVYYCARGGITGAPLVFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 451 aa, Artificial Sequence
  Synthetic
EVQLVESGGGWVQPGGSLRLSCAASGFTFSSHIMYWVRQAPGKGLEYVSGISSNGLSSYYVDSVKGRFTISRDNSKNLLYVHMGSLKPEDMAMYYCARGGRDRVPAVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKACPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 642 bp, Artificial Sequence
  Synthetic
gacatccagatgacccagtctccatcctccctgtctgcatctgtaggagacagagtcaccatcacttgccgggcaagtcagagcattagcaggtttttaaattggtatcagcagaaaccagggaaagcccctaaggtcctgatctatgttacatccagtttacaaagtggggtcccatcaaggttcagtggcagtggatctgggacagatttcactctcaccatcagcagtctgcaacctgaagattttgcaacttattactgtcaacagagttacaatacccctatcaccttcggccaagggacacgactggagattaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 70 - DNA, 642 bp, Artificial Sequence
  Synthetic
gacatccagatgacccagtctccatcctccctgtctgcatctataggagacagagtcaccatcacttgccgggcaagtcagagcattggcacctttttaaattggtatcaacaaaaaccagggaaagcccctaaggtcctgatctatgttgcatccagtttgcaaagtggggtcccatcaaggttcattggcagtggatctgggacagatttcactctcaccatcagcagtctgcaacctgaagattttgcaacttactactgtcaacagagttacagtgttccgatcaccttcggccaagggacacgactggagattaaacggactgtcgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 71 - DNA, 1353 bp, Artificial Sequence
  Synthetic
gaggtgcagctggtggagtctgggggaggcttggtccagcctggggggtccctgagactctcctgtgcagcctctggattcaccttcagtagttatgttatgcactgggtccgccaggctccagggaagggactggaatatgtttcaggtgttagtagtagtggggatagcacattttatgtagactctgtgaagggcagattcaccatctccagagacaattccaagaacacgctttatcttcaaatgggcagcctgagagctgaggacatggctgtgtattactgtgcgagagggggtataactggagctccactggtttttgatatctggggccaagggacaatggtcaccgtctcttcagcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 72 - DNA, 1353 bp, Artificial Sequence
  Synthetic
gaggtgcaactggtggagtctgggggaggctgggtccagccgggggggtccctgagactctcctgtgcagcctctggattcaccttcagtagtcatattatgtactgggtccgccaggctccagggaagggactggaatatgtttcgggtattagcagtaatggacttagctcatattatgttgactctgtgaagggcagattcaccatctccagagacaattccaagaatttactgtatgttcatatgggcagcctgaaacctgaggacatggctatgtattactgtgcgagagggggccgggatagagtgccagctgtctttgactactggggccagggaaccctggtcaccgtctcctccgcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagcctgcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa